(19) United States
(12) Patent Application Publication
(10) Pub. No.: US 2012/0041440 A1
(43) Pub. Date: Feb. 16, 2012

(54) INTRA-ORAL STABILISATION DEVICE

(75) Inventors: Darryl Chan Tong, Dunedin (NZ); John Neil Waddell, Dunedin (NZ); Murray Edward Fenton, Auckland (NZ)

(73) Assignee: OTAGO INNOVATION LIMITED, Dunedin (NZ)

(21) Appl. No.: 13/147,565

(22) PCT Filed: Feb. 3, 2010
(86) PCT No.: PCT/NZ10/00015

§ 371 Date: Oct. 17, 2011

Related U.S. Application Data
(60) Provisional application No. 61/149,822, filed on Feb. 4, 2009.

(51) Int. Cl. A61B 17/56

(52) U.S. Cl. 606/60

(57) ABSTRACT

An intra-oral stabilisation device has an upper tray (101), a lower tray a lower tray (201), and at least two spacers (217) to provide a spacing between the anterior portions of the upper and lower trays. Spacers (217) provide a spacing between anterior portions of the trays, and are positioned between corresponding posterior portions of the trays. Engagement features (301) are provided for engaging undersides of the posterior portions of the upper tray with upper sides of the posterior potions of the lower tray, and minimise lateral movement of the trays relative to one another once the trays are engaged with one another. In an alternative embodiment, the device may have only the lower tray, and may have a locator (219) for stabilising an endotracheal tube 401 relative to the lower tray. The upper or lower tray can be used to stabilise fracture segments of a fractured jaw.

### TECHNICAL FIELD

This invention relates to intra-oral stabilisation devices. Embodiments of the invention have application, for example, in stabilising one jaw relative to the other jaw, stabilising an endotracheal tube, and stabilising broken or fracture segments of a fractured jaw.

### BACKGROUND

Injuries to the maxillofacial (face and jaws) region are common in civilian practice, with the leading causes including interpersonal violence, motor vehicle incidents and sports. The lower jaw or mandible is one of the most common sites involved, ranging from 42-45% of all maxillofacial fractures—Kieser, J., Stephenson, S., Liston, P. N., Tong, D. T., and Langley, J. D., Serious facial fractures in New Zealand from 1979 to 1998, Int. J. Oral Maxillofac. Surg., (2002) 31: 206-209. In ballistic injuries from gunshot wound and/or fragment injuries from explosive devices in armed conflict, the face is second only to the extremities in terms of incidence of injury. Current literature shows 15-20% of all combat related injuries to involve the head, face and neck region—Lew, T. A., Walker, J. A., Wenke, J. C., Blackbourne, L. H., and Hale, R. G., Characterization of craniomaxillofacial battle injuries sustained by United Stales service members in the current conflicts of Iraq and Afghanastan, J. Oral. Maxillofac. Surg., (2010) 68:3-7. Furthermore, due to the current design of combat body armour offering more protection against torso injuries, more soldiers are surviving to have facial injuries treated resulting in a proportional increase in numbers of maxillofacial procedures.

Due to the fractures, the subject may experience significant bleeding and acute pain as the unstable fracture segments are able to move against each other. Furthermore, the subject may experience difficulties in swallowing, speaking, and controlling secretions. If the anterior mandible is severely fractured and unstable, potentially life-threatening acute airway obstruction may occur as the tongue is able to fall backwards and block the airway posteriorly. Unstable mandible fractures may also make jaw positioning for airway management—such as chin lift and jaw thrust—more difficult. The use of a face mask for bag-masking techniques in airway resuscitation, is also compromised with unstable anterior facial injuries or if significant portions of the jaw are missing altogether.

In order to stabilise jaw fractures, most current methods involve the wiring of teeth together to immobilise the jaws (intermaxillary fixation). This technique is difficult requiring high levels of advanced medical and dental training, potentially hazardous due to sharps injuries to the person performing the wiring and does not allow the jaws to be opened rapidly in cases of airway obstruction. The technique is also time consuming (both for insertion and removal), and requires the subject to be at least locally anaesthetised for both the wiring and the removal procedures. Insertion in skilled hands typically takes 30-45 minutes, and removal in skilled hands typically takes 30 minutes. The wires may snap, which would require additional time and suffering for the subject to be re-wired. Additionally, once the jaws are wired closed, the subject may be prevented from clearing respiratory secretions or vomit from his or her airway, which can compromise the subject's airway and potentially cause death from aspiration. If the nasal passages are obstructed due to trauma or post-surgical interventions including pack dressings, the subject may also experience difficulty in breathing through the mouth when the jaws are wired together. If the subject is obtunded or rendered unconscious, the airway compromise may potentially lead to fatal consequences. Some awake subjects may also experience a degree of claustrophobia due to the jaws being wired together.

These techniques are generally confined to well equipped hospitals, and do not lend themselves to pre-hospital treatment in the field (such as the battlefield or accident site for example), where services are limited or non-existent and that may rely on medical assistance from personnel who do not have the necessary training for performing wiring techniques.

U.S. Pat. No. 6,227,861 describes a splint device for stabilising a subject's mandible. That device comprises a substantially 2-dimensional body that is either attached to the subject's mandibular gums by wiring the device to the gums, or is attached to the mandible by brackets secured to the mandible or arch bars secured to the teeth. In each case, the device is time consuming to attach to the subject, and in the case of wiring, would require the subject to be anaesthetised for attachment and removal. Additionally, the subject could bite down on the splint with their top teeth, which could compromise the subject's ability to breathe through their mouth. Mouth breathing may be critical if nasal obstruction is present.

U.S. Pat. No. 6,086,365 describes a splint device for stabilising a subject's upper and lower jaws when his or her mandible is fractured, and that offers an alternative to surgical wiring devices. That device uses arch bands that are bonded to the upper and lower sets of teeth by dental cement. Metallic bands are linked around studs on the front faces of the bands, to immobilise the fracture. Due to the use of metallic bands, there is still the potential for sharps injuries to occur. Again, this device is time consuming to install and could not be used in a field environment with inadequate lighting and equipment as the cement must be applied to a dry surface. Additionally, once installed, the upper and lower jaws are closed together, which may again pose the same difficulties as previously mentioned regarding potential airway issues and aspiration risk.

An alternative device is the IMF RAPID device supplied by Synthes North America. That device utilises flexible bands that are wrapped around individual teeth that support anchorage points. A polymeric band is then zig-zagged between the anchors: Due to the use of the polymeric band, this device avoids the risk of sharps injuries. However, as the flexible bands need to be wrapped around individual teeth, again the device is time consuming to install. Once installed, the upper and lower jaws are closed together, which may again pose the same difficulties as previously mentioned regarding potential airway issues and aspiration risk. Additionally, the device is time consuming to remove, requiring the bands to be removed from the individual teeth.

When a subject is semi-conscious or unconscious, the subject may requite endotracheal intubation to maintain airway patency and protection. It is important that the endotracheal tube is inserted and maintained in a correct position to provide adequate airway patency and protection within the trachea, with prevention of the endotracheal tube from migrating or being dislodged or pulled out completely being paramount. Migration of the endotracheal tube deeper into the airway Poses significant dangers due to the endotracheal tube potentially passing into a main stem bronchus beyond the carina, which is the point of bifurcation of the lower airways into each lung, resulting in only one lung being ventilated with resulting morbidity. The endotracheal tube tip should be 10-15 mm above the carina in order to ventilate both lungs. One conventional technique of positioning an endotracheal tube comprises externally fixing the tube to the subject with surgical tape such as LEUKOPLAST SLEEK by Smith & Nephew or trach ties, adhesive, or straps. This technique is impractical if the subject is suffering from significant facial soft tissue injuries such as facial burns or multiple lacerations for example. Additionally, when an endotracheal tube is positioned in such a manner, there is nothing to prevent a subject from biting down on the-tube. Biting down on the tube could prevent adequate ventilation, which could potentially result in death.

An alternative technique for inserting an endotracheal tube comprises inserting the tube via a surgical airway in the user's neck. This technique is surgically invasive and is only used in emergency situations when conventional endotracheal intubation is impractical, unavailable or impossible to achieve. The subject may or may not receive any anaesthetic before insertion of the tube into this surgical airway access. The tube will also need some type of additional external fixation to prevent the tube from migrating or being dislodged.

U.S. Pat. No. 5,626,128 describes a device for maintaining an endotracheal tube in a subject's mouth without external fixation. The device has a tray that covers the subject's upper teeth and the roof of the mouth. A guide member is positioned on the front of the tray to receive an endotracheal tube. In one embodiment of the device, there is nothing to prevent a subject from biting up on the tube with their lower front teeth. The tube is unprotected and potentially can be crushed, thereby preventing adequate ventilation which could potentially result in death.

In an alternative embodiment, the tray is wedge-shaped, with the thickest part of the tray positioned at the rear of the tray. While that configuration allegedly prevents a subject from biting down on the tube, it creates a fulcrum which would allow an unstable maxilla (as in a complete Le Fort I or II midface injury) to pivot as pressure is placed on the device from the subject's posterior dentition. This would also lead to the maxillary fracture opening anteriorly, potentially causing an increase in pain and bleeding and decreasing the space between the jaws and dentition anteriorly as the maxilla tips downwards at the front. Due to the configuration of that device, the device can only be used on the upper jaw, as the portion that covers the roof of the mouth would interfere with the tongue if the device was used on the bottom jaw.

It is undesirable to fix an endotracheal tube to the upper jaw, as the endotracheal tube has a pre-fabricated downward curve which facilitates easier placement due to an arc of insertion into the airway via laryngoscopy. By fixing the endotracheal tube rigidly to the maxilla, the curve of the tube may be straightened which may cause the tube to kink, may dislodge the tube from its optimal position within the trachea and may physically be in the way of the surgeon.

Therefore, a need exists for a simple-to-use device that can provide fracture stability and/or temporary intermaxillary stabilisation prior to transfer or evacuation for specialist care. A need also exists for a simple to use device that can provide a reliable platform for airway management by providing endotracheal tube security, especially during evacuation or transfer of the subject.

It is an object of at least preferred embodiments of the invention to provide an intra-oral stabilisation device that addresses one or more of the disadvantages outlined above. It is an alternative object of at least preferred embodiments of the present invention to provide the public with a useful alternative.

In this specification where reference has been made to patent specifications, other external documents, or other sources of information, this is generally for the purpose of providing a context for discussing the features of the invention. Unless specifically stated otherwise, reference to such external documents or such sources of information is not to be construed as an admission that such documents or such sources of information, in any jurisdiction, are prior art or form part of the common general knowledge in the art.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or non-limiting embodiments identified in this Brief Summary, which is included for purposes of illustration only and not restriction.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In accordance with a first aspect of the present invention, there is provided a device for stabilising a jaw relative to the other jaw, the device comprising: an upper tray for installing onto the upper dentition of a subject, the upper tray having an anterior portion and two posterior portions; a lower tray for installing onto the lower dentition of the subject, the lower tray having an anterior portion and two posterior portions; at least two spacers to provide a spacing between the anterior portions of the upper and lower trays when installed on the upper and lower dentitions of the subject, wherein at least one spacer is provided between corresponding posterior portions on one side of the upper and lower trays and at least one other spacer is provided between corresponding posterior portions on the other side of the upper and lower trays; and engagement features for engaging undersides of the posterior portions of the upper tray with upper sides of the posterior portions of the lower tray, and that minimise lateral movement of the trays relative to one another once the posterior portions of the trays are engaged with one another.

In this and the following aspects, the dentitions may be full dentitions or partial dentitions. That is, the dentitions may or may not have full sets of teeth. The device will be equally suitable for use in a fully dentate subject or when one or more teeth are missing from one or both of the dentitions, which makes the device suitable for use on a blast victim for example. The device may be suitable for use when the dentitions are edentulous (have no teeth).

In an embodiment of the invention, the engagement features provide a plurality of possible forward-to-rearward engagement positions of the trays, and once engaged, minimise forward-to-rearward movement of the trays relative to one another. In an embodiment of the invention, the engagement features, once engaged, substantially prevent forward-to-rearward movement of the trays relative to one another.

In an embodiment of the invention, the engagement features provide a plurality of possible side-to-side engagement positions of the trays, and once engaged, minimise side-to-side or lateral movement of the trays relative to one another. In an embodiment of the invention, the engagement features, once engaged, substantially prevent side-to-side movement of the trays relative to one another.

In an embodiment of the invention, the engagement features enable the trays to be engaged to one another, and for lateral movement between the trays to be minimised, in a plurality of relative angular positions of the trays.

In an embodiment of the invention, an engagement feature is provided on each of the posterior portions of the upper tray, and a further engagement feature is provided on each of the posterior portions of the lower tray, to engage the posterior portions of the trays with one another. The engagement features may comprise a plurality of members on each of the posterior portions of the upper tray, which members are engageable with a plurality of members on each of the posterior portions of the lower tray. The members on one tray may comprise projections, and the members on the other tray may comprise recesses. Alternatively, each tray may comprise projections and/or recesses that engage with one another. The plurality of recesses/projections will provide the plurality of forward-to-rearward and/or side-to-side engagement positions of the trays.

In one form, the engagement features may comprise hook and loop fasteners, such as those marketed under the trade mark VELCRO or similar, where a plurality of hooks on one tray will be engageable with a plurality of loops on the other tray. In another form, the members on the upper tray may comprise enlarged heads that engage with enlarged heads of the members on the lower tray. A suitable product is marketed under the trade mark DUAL LOCK by 3M:

In an alternative form, the engagement features may each comprise a single member on one of the trays that engages with a single member on the other of the trays. Alternatively, the engagement features may each comprise a projection on one of the trays that is selectively engageable with a plurality of recesses on the other of the trays.

In an alternative embodiment of the invention, on a left posterior portion of the device, an engagement feature is provided on one of the underside of the upper tray and the upper side of the lower tray, and on a right posterior portion of the device, an engagement feature is provided on one of the underside of the upper tray and the upper side of the lower tray. In this embodiment, the engagement features may for example comprise adhesive surfaces that are provided on one or both of the trays.

In an embodiment of the invention, the engagement features are disengageable so the upper and lower trays can be manually moved apart if necessary, but the engagement features will engage with sufficient strength that the upper and lower trays will not inadvertently separate when engaged and the trays are installed on a subject's upper and lower dentition.

In an embodiment of the invention, the spacers provide a sufficient spacing between the anterior portions of the upper and lower trays when installed on the upper and lower dentitions of the subject that an endotracheal tube can be inserted between the anterior portions of the trays.

The spacers may be provided on one or both of the upper and lower trays.

In an embodiment of the invention, the spacers each comprise a forwardly inclined ramp. The ramps will enable the subject's rear teeth to be in closer proximity to one another than the subject's front teeth, when the trays are installed on the upper and lower dentitions of the subject. The ramps may comprise ribs of varying heights.

In an embodiment of the invention, the ramps arc provided on the lower tray, and each ramp has an arcuate surface that faces the upper tray in use, that has a convex curvature when viewed from the upper tray, and that is curved in a forward/rearward direction of the device.

In an embodiment of the invention, the lower tray comprises two forwardly inclined ramps, and the ramps each terminate at or near the anterior portion of the tray to form a shoulder, and the shoulders form a spacing in the anterior portion of the lower tray that is sufficient to receive part of an endotracheal tube.

The engagement features may be provided on the spacers.

In an embodiment of the invention, the lower tray comprises a locator for receipt of part of an endotracheal tube, to assist with maintaining the tube in a desired position in use. The locator may comprise a recess formed in a surface of the lower tray that faces the other of the trays when mounted to a subject's upper and lower dentition. The recess may be arcuate, to support an endotracheal tube with a curved outer periphery. The locator may be provided in an anterior portion of the lower tray, so the locator can be readily accessed from the front of the subject's mouth. The locator may comprise a frictional, textured, or adhesive surface for minimising movement of an endotracheal tube when received in the locator.

In an embodiment of the invention, the lower tray comprises a fastener-receiving region that is configured for receipt of a fastener for fastening the endotracheal tube to the locator, to minimise or prevent unwanted movement of the endotracheal tube once inserted. The fastener-receiving region may be provided on an anterior portion of the lower tray, and may be provided on an external anterior portion so the fastener-receiving region can be readily accessed from the front of a subject's mouth. The fastener-receiving region may comprise a support for receipt of a fastener, to minimise or prevent unwanted movement of an endotracheal tube once inserted. The support may be suitable for receipt of any suitable type of fastener, such as a tie or strap that can partly surround the tube for example.

The device may be provided in combination with at least one suitable fastener. In an embodiment of the invention, the fastener is readily disengageable from the fastener receiving region to enable removal of the endotracheal tube.

In an embodiment of the invention, at least one of the trays comprises at least one flexible region, to enable the tray to be mounted to an asymmetric dental arch (such as an arch that has been subjected to trauma for example) or to dental arches of different sizes. In an embodiment of the invention, the flexible region(s) comprise(s) a hinge or pivot. In an embodiment of the invention, the tray(s) comprising the flexible region(s) further comprise(s) a relatively rigid anterior portion for supporting a subject's front teeth, two relatively rigid posterior portions for supporting a subject's side and posterior teeth on respective sides of the dental arch, and a flexible region between the anterior portion and each of the posterior portions. In that embodiment, the flexible region(s) may comprise a hinge. Typically, six teeth, or the section of the dentition corresponding to the six front teeth if the teeth are missing, would be positioned in the region forward of the hinges.

In an embodiment of the invention, the trays are moulded plastic trays, and the flexible region(s) comprise a hinge that is incorporated into the trays as part of the moulding process. Each tray may have flexible region(s). Each tray may have two flexible regions.

In an embodiment of the invention, the engagement features are configured to accommodate a plurality of relative adjusted widths of the posterior portions of the trays, but to engage the trays to one another and minimise lateral movement of the trays once engaged.

In an embodiment of the invention, the anterior region of each tray is configured to substantially enclose a subject's front teeth, and comprises an outer wall that is configured to extend over the front of the teeth, a base wall that is configured to extend over the ends of the teeth, and an inner wall that is configured to extend over the rear of the teeth. The outer wall, base wall, and inner wall may form a substantial U shape in cross-section. In an embodiment of the invention, the posterior portions are configured to substantially enclose a subject's side and rear teeth, and each comprise an outer wall that is configured to extend over the outer surface of the teeth, a base wall that is configured to extend over the ends of the teeth, and an inner wall that is configured to extend over the rear of the teeth. In an embodiment of the invention, in each posterior portion, the outer wall, base wall, and inner wall form a substantial U shape in cross-section.

In accordance with a second aspect of the present invention, there is provided a kit of parts for a device as outlined in relation to the first aspect above, the kit comprising: an upper tray for installing onto the upper dentition of a subject, the upper tray having an anterior portion and two posterior portions; a lower tray for installing onto the lower dentition of the subject, the lower tray having an anterior portion and two posterior portions; at least two spacers to provide a spacing between the anterior portions of the upper and lower trays when installed on the upper and lower dentitions of the subject, wherein a spacer is provided on a posterior portion of at least one of the trays and a spacer is provided on the other posterior portion of at least one of the trays; and engagement features for engaging undersides of the posterior portions of the upper tray with upper sides of the posterior portions of the lower tray, and that are positioned on or adapted to be positioned on one or both of the trays.

The engagement features may be pre-attached to or formed as part of the tray(s). Alternatively, the engagement features may initially be separate from the trays in the kit.

In an embodiment of the invention, the kit further comprises a substance for use in mounting the trays to a subject's dentitions. The substance may be a substance that will form a mould around the teeth of the subject's dentitions, to mount the trays to the subject's teeth and thereby enhance the attachment to the trays to the teeth. The substance may be a silicone-based substance for example.

In an embodiment of the invention, the kit further comprises an applicator for the substance.

In an embodiment of the invention, the kit further comprises a fastener for fastening an endotracheal tube to a tube locator on one of the trays.

The kit may comprise a plurality of upper trays of different sizes and a plurality of lower trays of different sizes.

The kit may further comprise instructions for use.

In accordance with a third aspect of the present invention, there is provided a method of stabilising a subject's jaw relative to their other jaw, comprising: providing a device as outlined in relation to the first aspect above; installing the upper tray onto the subject's upper dentition; installing the lower tray onto the subject's lower dentition; and engaging the posterior portions of the upper and lower trays to one another to minimise lateral movement of the trays relative to one another and to maintain a spacing between the anterior portions of the upper and lower trays.

In an embodiment of the invention, the step of engaging the posterior portions substantially prevents lateral movement between the upper and lower trays.

The method may comprise, prior to engaging the posterior portions, attaching the engagement features to one or both of the trays. Alternatively, the engagement features may be pre-attached or integrally formed with the tray(s).

The method may comprise, prior to engaging the posterior portions, moving the lower jaw forward relative to the upper jaw or moving the upper jaw forward relative to the lower jaw.

The steps of installing the upper and lower trays may comprise applying a substance to the interiors of the trays to enhance the attachment of the trays to the subject's teeth. The substance may be a substance that will form a mould around the teeth of the subject's dentitions, to mount the trays to the subject's teeth and thereby enhance the attachment to the trays to the teeth. The substance may be a silicone-based substance for example.

The steps of installing the upper and lower trays may comprise adjusting the width of the dental trays to fit to the dentitions of the subject.

The method may comprise inserting an endotracheal tube down the subject's throat, and positioning part of the endotracheal tube between the anterior portions of the upper and lower trays. The endotracheal tube may be inserted down the subject's throat prior to installing the upper and lower trays. In that embodiment, part of the endotracheal tube will be positioned between the anterior portions of the upper and lower trays during installation of the upper and lower trays. Alternatively, the endotracheal tube may be inserted down the subject's throat, and part of the endotracheal tube positioned between the anterior portions of the upper and lower trays, after installation of the upper and lower trays.

The method may comprise fastening the endotracheal tube to the lower tray.

When used with a subject having at least one fractured jaw comprising fracture segments, the step of installing the tray(s) onto the subject's dentition(s) corresponding to the fractured jaw(s) may comprise initially moving the fracture segments so that the fracture segments are generally aligned, and the installed tray(s) minimise(s) relative movement of the fracture segments. The anterior portion(s) of the tray(s) corresponding to the fractured jaw(s) may be relatively rigid, and the method may comprise installing the relatively rigid anterior portion(s) of the tray(s) onto the teeth corresponding to the front of the jaw. The jaw may be the upper jaw or the lower jaw, and accordingly the tray may be the upper tray or the lower tray.

When the upper and lower jaws are angularly misaligned, the method may comprise engaging the engagement feature(s) without correcting the angular misalignment.

In accordance with a fourth aspect of the present invention, there is provided a device for stabilising an endotracheal tube relative to a jaw, the device comprising: a tray for installing onto the lower dentition of the subject, the tray having an anterior portion and two posterior portions; a forwardly inclined ramp positioned on each posterior portion of the tray, with each ramp terminating at or near the anterior portion of the tray to form a shoulder, wherein the shoulders define an opening at the anterior portion of the tray that is of a sufficient size to enable an endotracheal tube to extend therethrough; and a locator for receiving a portion of an endotracheal tube.

In an embodiment of the invention, the ramps terminate at the anterior portion of the tray.

In an embodiment of the invention, the ramps comprise a plurality of ribs of varying heights. The ribs may be integrally formed with the ramps.

The locator may comprise a tongue that extends from the anterior portion of the tray to substantially align with the opening at the anterior portion.

The locator may comprise a concave receiving surface for receiving and corresponding with a portion of the endotracheal tube. The concave receiving surface may comprise a frictional, textured, or adhesive surface for minimising movement of an endotracheal tube when received in the locator.

In an embodiment of the invention, the device further comprises a fastener for attaching an endotracheal tube to the locator.

The tray may comprise at least one flexible region, to enable the tray to be mounted to an asymmetric dental arch or to dental arches of different sizes. In an embodiment of the invention, the tray comprises a relatively rigid anterior portion for supporting a subject's front teeth, two relatively rigid posterior portions for supporting a subject's side and posterior teeth on respective sides of the dental arch, and a flexible region between the anterior portion and each of the posterior portions. Each ramp may terminate at a respective one of the flexible regions to form the shoulders.

In accordance with a fifth aspect of the present invention, there is provided the combination of a device as outlined in relation to the fourth aspect above and an endotracheal tube, wherein the height of the shoulders is at least as high as a diameter of the endotracheal tube.

In accordance with a sixth aspect of the present invention, there is provided a kit for stabilising an endotracheal tube relative to a jaw, the kit comprising: a device as outlined in relation to the fourth aspect above; and a substance for use in mounting the tray to a subject's dentition.

The substance may be a substance that will form a mould around the teeth of the subject's dentition, to mount the tray to the subject's teeth and thereby enhance the attachment to the tray to the teeth. The substance may be a silicone-based substance for example.

In an embodiment of the invention, the kit further comprises an applicator for the substance.

In an embodiment of the invention, the kit further comprises a plurality of trays of different sizes.

In an embodiment of the invention, the kit comprises a plurality of fasteners.

The kit may further comprise instructions for use.

In accordance with a seventh aspect of the present invention, there is provided a method of stabilising an endotracheal tube relative to a jaw of a subject, comprising: providing a device as outlined in relation to the fourth aspect above; installing the tray onto the subject's lower dentition; inserting an endotracheal tube down the subject's throat; and positioning part of the endotracheal tube in the opening at the anterior portion of the tray.

The endotracheal tube may be inserted down the subject's throat prior to installing the tray. In that embodiment, part of the endotracheal tube may be positioned in the opening at the anterior portion of the tray during or after installation of the tray. Alternatively, the endotracheal tube may be inserted down the subject's throat, and part of the endotracheal tube positioned in the opening at the anterior portion of the tray, after installation of the tray. In that embodiment, part of the endotracheal tube may be positioned in the opening at the anterior portion of the tray concurrently with the insertion of the endotracheal tube down the subject's throat or after the insertion of the endotracheal tube down the subject's throat.

The method may further comprise fastening the endotracheal tube to the tray.

In an embodiment of the invention, the step of installing the tray onto the subject's lower dentition comprises applying a substance to the interior of the tray to enhance the attachment of the tray to the subject's teeth. The substance may be a substance that will form a mould around the teeth of the subject's dentition, to mount the tray to the subject's teeth and thereby enhance the attachment to the tray to the teeth. The substance may be a silicone-based substance for example.

When used with a subject having a fractured lower jaw comprising fracture segments, the step of installing the tray onto the subject's lower dentition may comprise initially moving the fracture segments so that adjacent bone ends of the fracture segments are generally aligned, and the installed tray minimises relative movement of the fracture segments.

In accordance with an eighth aspect of the present invention, there is provided a method of stabilising a subject's fractured jaw comprising fracture segments, the method comprising: providing a tray for installing onto the dentition of the subject corresponding to the fractured jaw, the tray having an anterior portion and two posterior portions; and installing the tray onto the subject's dentition so that the installed tray minimises relative movement of the fracture segments.

The jaw may be a lower jaw, an upper jaw, or both a lower jaw and an upper jaw.

When adjacent bone ends of the fracture segments are misaligned, the method may comprise, prior to installing the tray onto the subject's dentition, generally aligning the bone ends. When adjacent bone ends of the fracture segments are aligned or generally aligned, the tray can be installed onto the subject's dentition without moving the fracture segments.

The step of installing the tray may comprise applying a substance to the interior of the tray to enhance the attachment of the tray to the subject's teeth. The substance may be a substance that will form a mould around the teeth of the subject's dentition, to mount the tray to the subject's teeth and thereby enhance the attachment to the tray to the teeth. The substance may be a silicone-based substance for example.

In an embodiment of the invention, the tray comprises a relatively rigid anterior portion, and the method comprises installing the relatively rigid anterior portion of the tray onto the teeth corresponding to the front of the jaw.

In an embodiment of the invention, the tray comprises an anterior portion, two posterior portions, and a forwardly inclined ramp that is positioned on each posterior portion of the tray, with each ramp terminating at the anterior portion of the tray to form a shoulder, with the shoulders defining an opening at the anterior portion of the tray that is of a sufficient size to enable an endotracheal tube to extend therethrough, and wherein the tray further comprises a locator for receiving a portion of an endotracheal tube

The tray(s) used in the eighth aspect may have any one or more of the features outlined in relation to the first or fourth aspects above.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Where specific integers are mentioned herein which have known equivalents in the art to which this invention relates, such known equivalents are deemed to be incorporated herein as if individually set forth.

The invention consists in the foregoing and also envisages constructions of which the following gives examples only.

### BRIEF DESCRIPTION OF THE DRAWINGS

The invention may be more fully understood by way of example only having reference to the accompanying drawings wherein:

FIG. 1 is a front overhead perspective view of a first embodiment device for stabilising a jaw relative to the other jaw;

FIG. 2 is a front underside perspective view of the device of FIG. 1;

FIG. 3 is a right side view of the device of FIG. 1;

FIG. 4 is a rear view of the device of FIG. 1;

FIG. 5 is an underside view of the device of FIG. 1;

FIG. 6 is a front perspective view of the device of FIG. 1;

FIG. 7 is a front overhead perspective view of the device of FIG. 1;

FIG. 8 is a front overhead perspective view from the other side;

FIG. 9 is a rear underside perspective view of the upper tray of the device of FIG. 1;

FIG. 10 is an overhead rear perspective view of the upper tray of the device of FIG. 1;

FIG. 11 is an underside view of the upper tray of the device of FIG. 1;

FIG. 12 is an overhead perspective view of the upper tray of the device of FIG. 1;

FIG. 13 is a rear view of the upper tray of the device of FIG. 1;

FIG. 14 is a right side view of the upper tray of the device of FIG. 1;

FIG. 15 is a front view of the upper tray of the device of FIG. 1;

FIG. 16 is a front underside perspective view of the lower tray of the device of FIG. 1;

FIG. 17 is an overhead front perspective view of the lower tray of the device of FIG. 1;

FIG. 18 is an underside view of the lower tray of the device of FIG. 1;

FIG. 19 is an overhead view of the lower tray of the device of FIG. 1;

FIG. 20 is a rear view of the lower tray of the device of FIG. 1;

FIG. 21 is a right side view of the lower tray of the device of FIG. 1;

FIG. 22 is a front view of the lower tray of the device of FIG. 1;

FIG. 23a is a schematic end or side view of an engagement feature of the device of FIG. 1, with the two portions of the engagement feature separated;

FIG. 23b is a view similar to FIG. 23a, but with the two portions engaged;

FIG. 24 is an overhead perspective view showing the device of FIG. 1 and its ability to accommodate angular or lateral misalignment and mandibular advancement;

FIG. 25 is a front perspective view of the device of FIG. 1 in a similar position to FIG. 24;

FIG. 26a is a schematic view showing the first step in a method of using the device of FIG. 1 with a subject;

FIG. 26b shows a second step in the method, with the lower tray placed onto the lower dentition of the subject;

FIG. 26c shows a third step in the method, with the upper tray placed on the upper dentition of the subject;

FIG. 26d shows a fourth step in the method, with the upper and lower trays engaged to one another and an endotracheal tube installed in the device;

FIG. 27 is an exploded perspective view of the components of the device of FIG. 1;

FIG. 28 is a front perspective view of a second, alternative embodiment device;

FIG. 29a shows a fractured jaw that can be stabilised using one of the devices; and

FIG. 29b shows the application of the device of FIG. 28 to stabilise the fractured jaw.

### DETAILED DESCRIPTION OF THE INVENTION

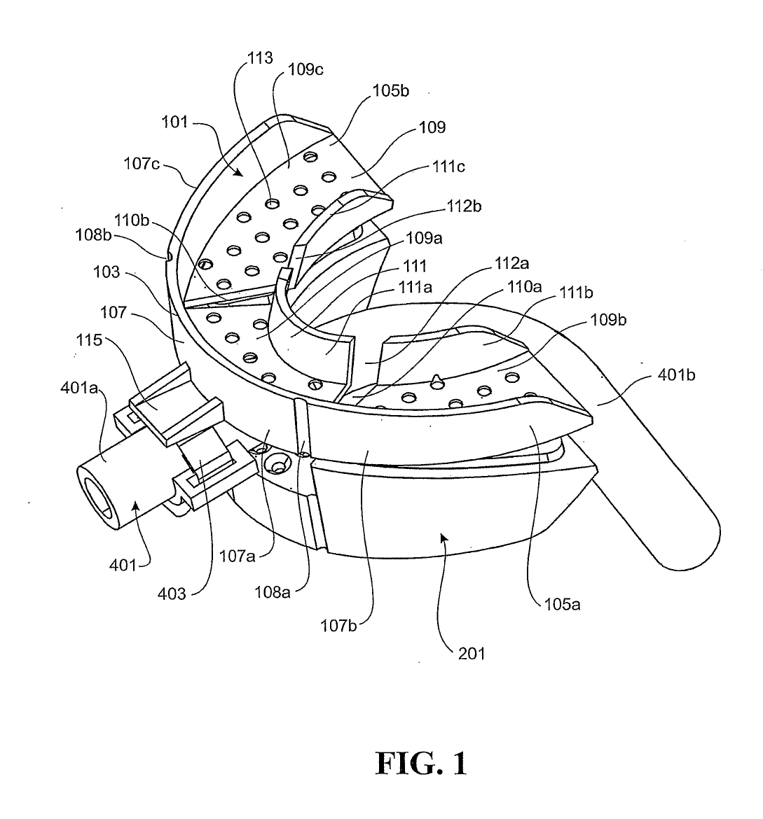

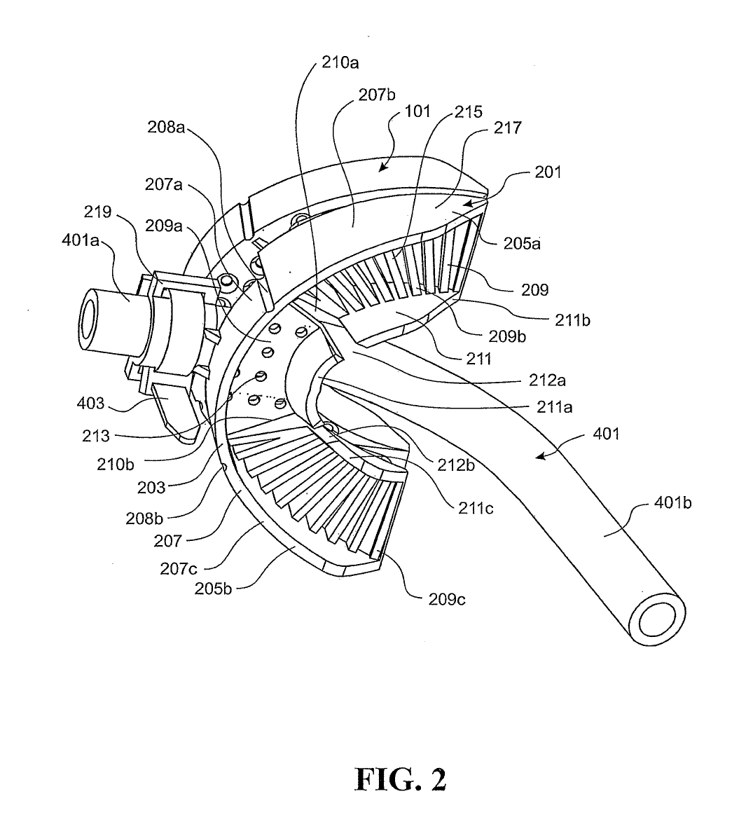

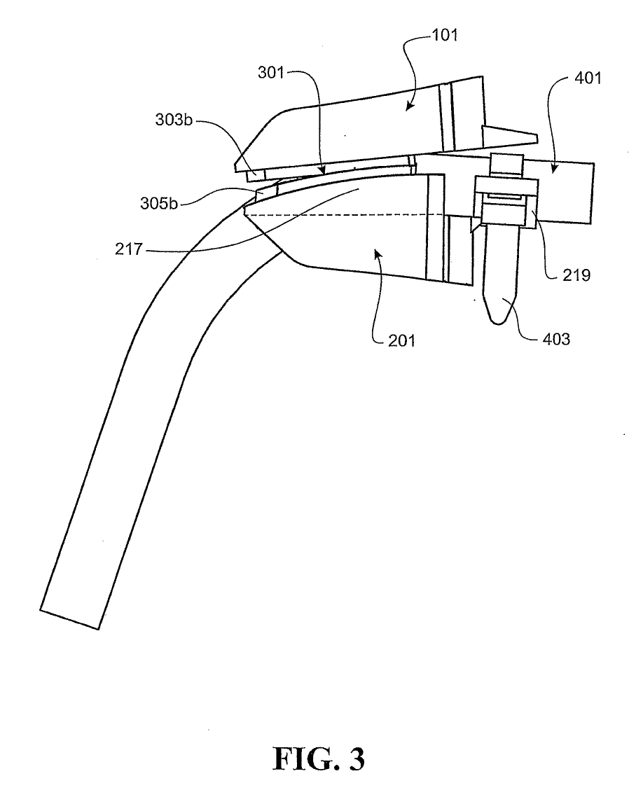

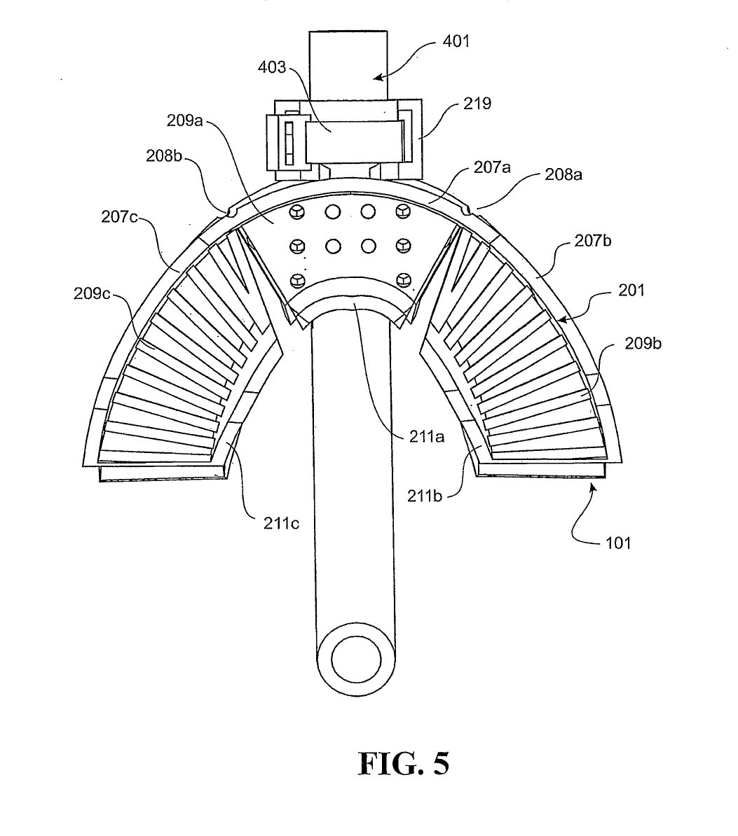

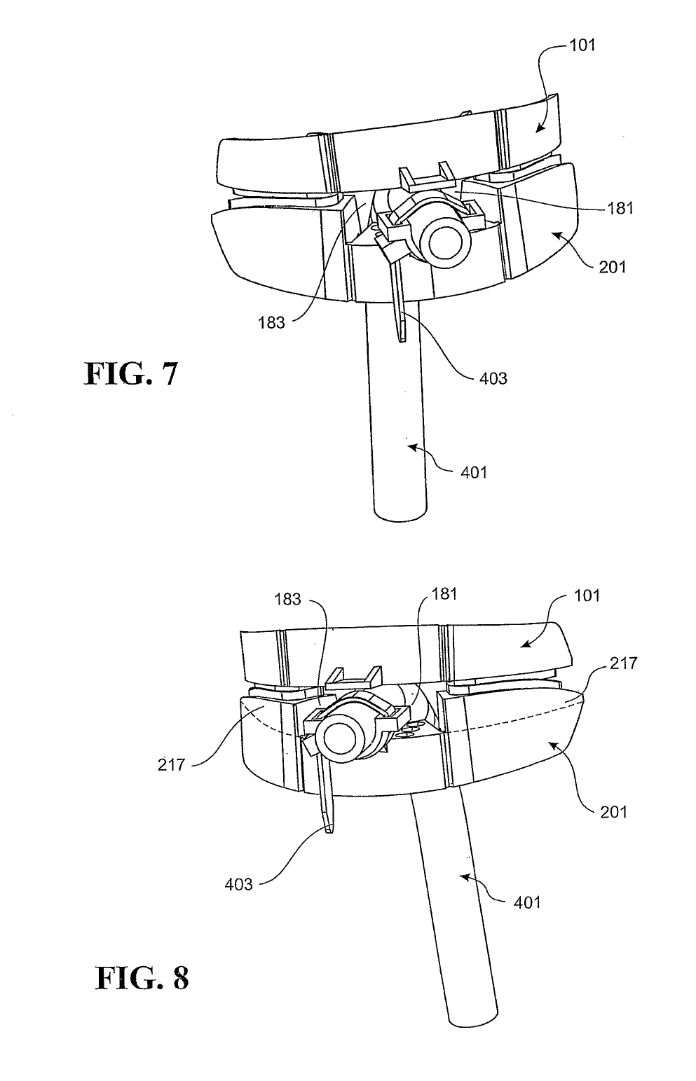

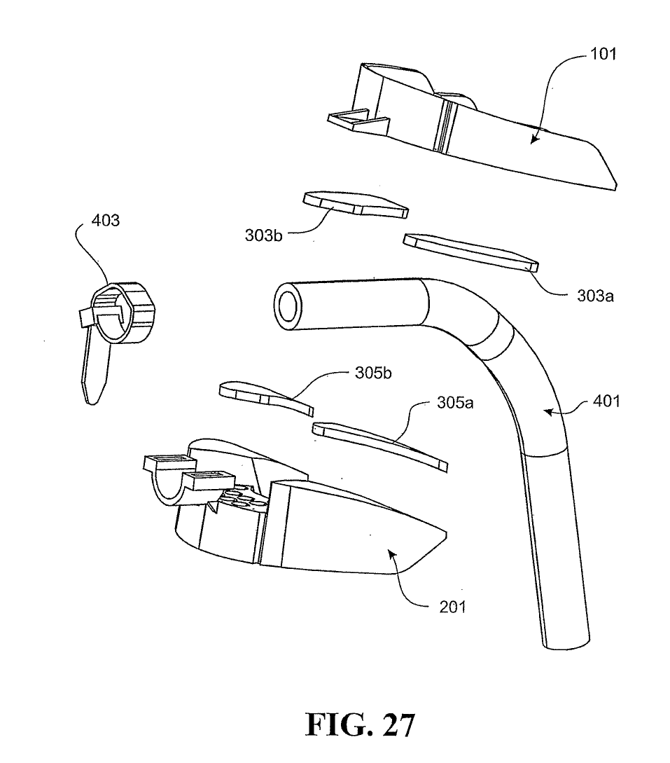

An intra-oral stabilisation device of a first embodiment of the invention is shown in FIGS. 1 to 8, and in exploded view in FIG. 27. The device comprises an upper tray 101 for installing onto the upper dentition of a subject, the upper tray having an anterior portion 103, a left side posterior portion 105a, and a right side posterior portion 105b. The device further comprises a lower tray 201 for installing onto the lower dentition of the subject, the lower tray having an anterior portion 203, a left side posterior portion 205a, and a right side posterior portion 205b.

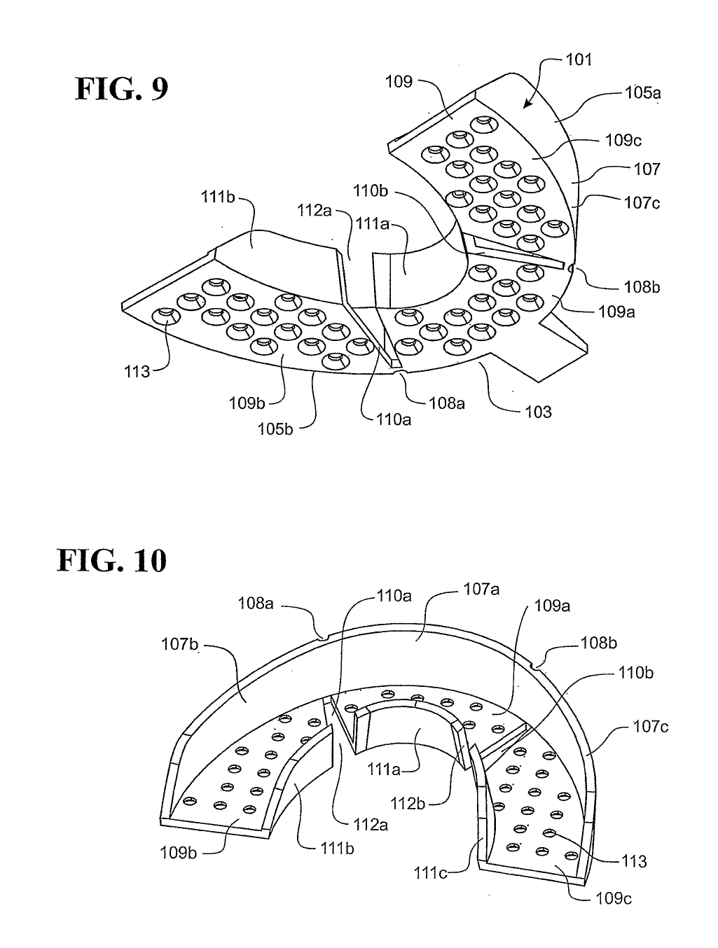

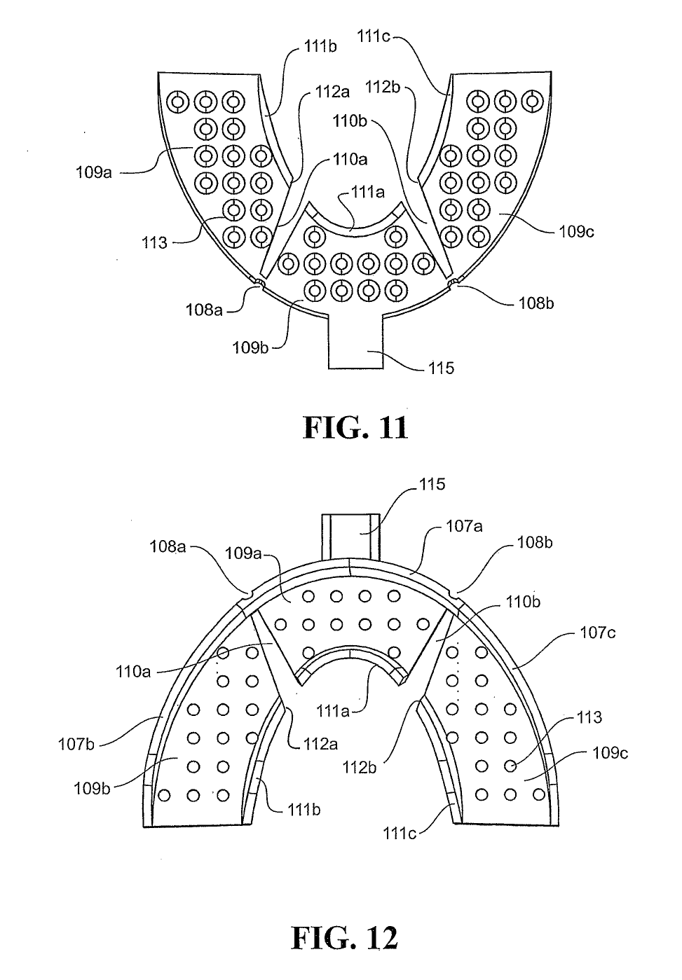

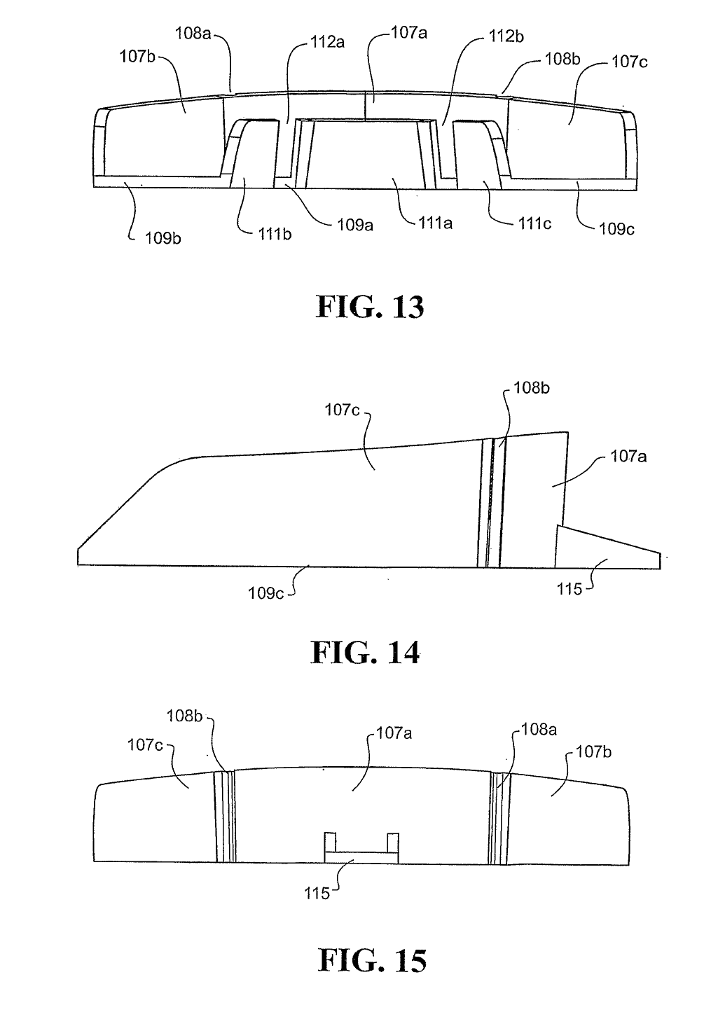

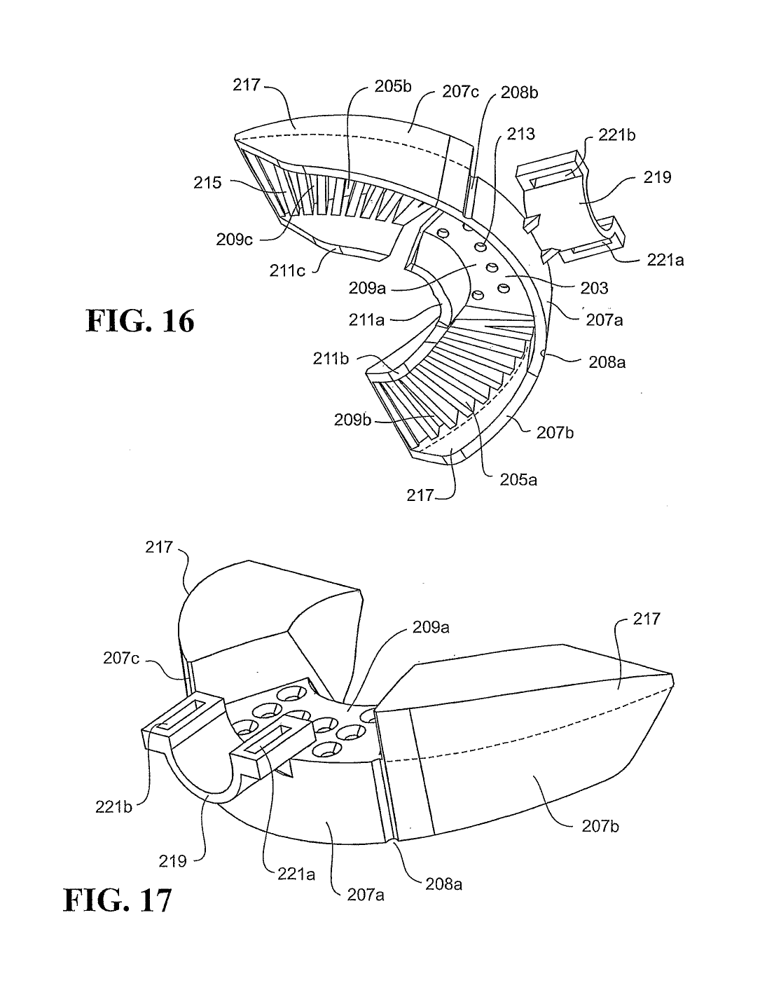

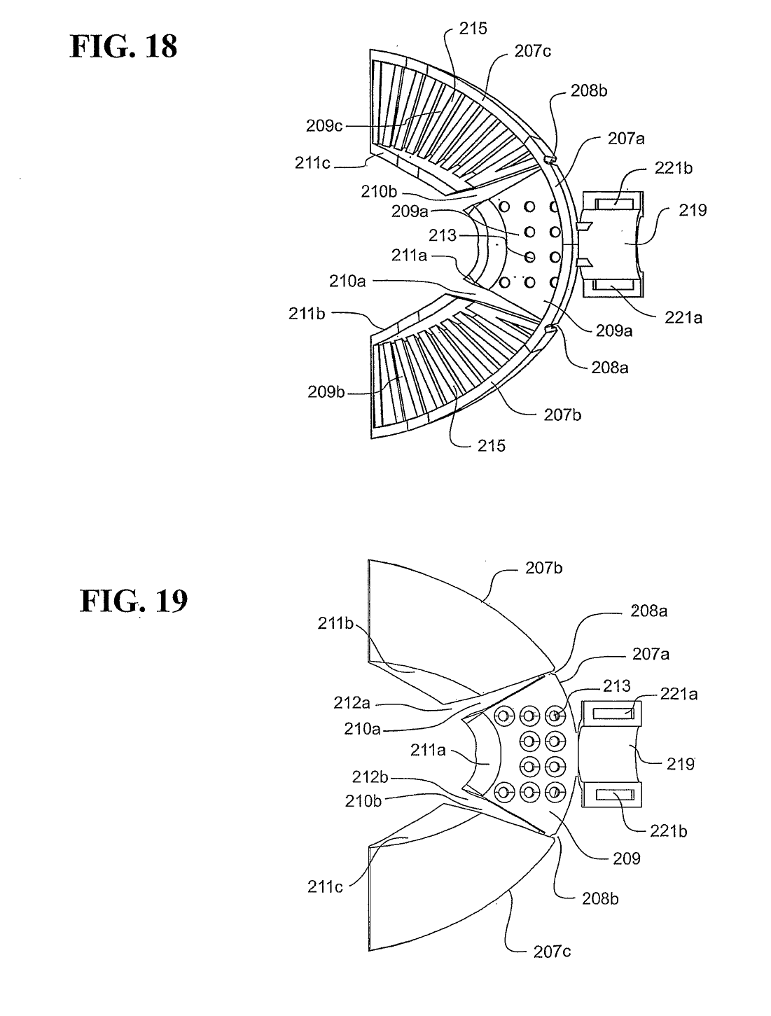

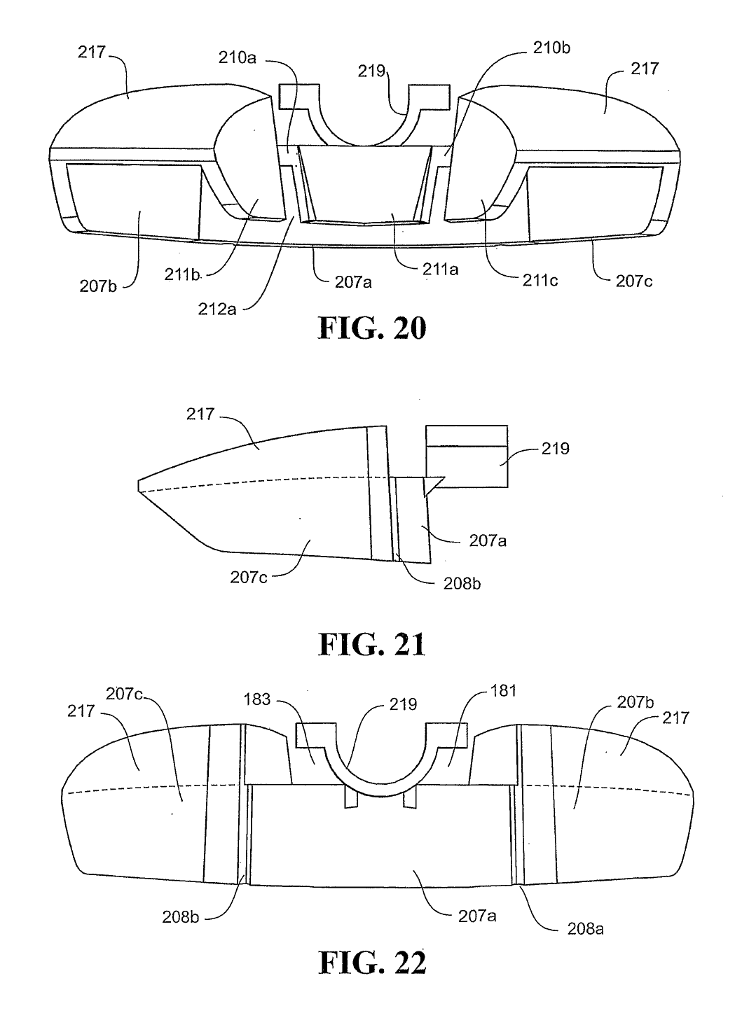

The upper tray is shown in detail in FIGS. 9 to 15, and the lower tray is shown in detail in FIGS. 16 to 22.

The anterior portion 103, 203 of each of the upper and lower frays is configured to receive and enclose the front teeth of a typical human subject's upper and lower dentitions respectively. The dentitions may be full dentitions or partial dentitions. That is, the dentitions may or may not have full sets of teeth. The device may be suitable for use when one or more teeth are missing from one or both of the dentitions, which makes the device suitable for use on a blast victim for example.

The device may also be suitable for use when the dentition(s) is/are edentulous (have no teeth). If all teeth have been removed previously and the subject was a denture wearer, the device will be usable depending on the height of the residual ridges and degree of the undercuts of the ridges. Provided there is sufficient ridge height and undercuts, then the device will be usable. If the subject has lost their teeth due to a blast injury for example, then as long as there is sufficient ridge height, the device will be usable as the ridges will have a degree of undercut.

The left side posterior portion 105a, 205a of each of the upper and lower trays is configured to receive the teeth that are rearward of the front teeth on the left side of the subject's mouth, and the right side posterior portion 105b, 205b of each of the upper and lower trays is configured to receive the teeth that ate rearward of the front teeth on the right side of the subject's mouth.

With reference to FIGS. 9 to 15, the anterior portion 103 of the upper tray is configured to enclose a subject's front six teeth, or the section of the dentition corresponding to the subject's six front teeth if those teeth are missing, and comprises an outer wall 107a that is configured to extend over the front of the teeth, a base wall 109a that is configured to extend over the ends of the teeth, and an inner wall 111a that is configured to extend over the rear of the teeth. The outer wall 107a, base wall 109a, and inner wall 111a form a substantial U-shape in cross-section. The posterior portions 105a, 105b are configured to enclose a subject's side and rear teeth, and comprise outer walls 107b, 107c that are configured to extend over the outer surface of the teeth, base walls 109b, 109c that are configured to extend over the ends of the teeth, and inner walls 111b, 111c that are configured to extend over the rear of the teeth. The outer walls 107b, 107c, base walls 109b, 109c, and inner walls 111b, 111c form respective substantial U-shapes in cross-section.

In plan view, the upper tray is substantially U-shaped, with the region behind the inner wall being open. That enables the tray to fit to the upper dentition of a variety of subjects having mouths of different sizes and shapes. It can be seen that the lower tray is also substantially U-shaped, with the region behind the inner wall being open. That enables the tongue to remain unobstructed. The open trays also enable the trays to flex, as described below.

The upper tray comprises at least one flexible region, to enable the tray to be mounted to an asymmetric dental arch, or to arches of different sizes. In the form shown, the upper tray has two flexible regions defined by hinges 108a, 108b. The tray may be a moulded item, and the hinges 108a, 108b may be formed into the tray as part of the moulding process. The flexible regions separate the anterior portion 103 of the tray from the posterior portions 105a, 105b of the tray, and are formed at a point of maximum curvature of the tray. Anterior base wall 109a is separated from the posterior base walls 109b, 109c by respective tapered recesses 110a, 110b, and anterior rear wall 111a is separated from the anterior rear walls 111b, 111c by respective recesses 112a, 112b.

The hinges 108a, 108b and the recesses 110a, 110b, 112a, 112b enable the posterior portions 105a, 105b of the tray to be flexed inwardly toward'one another (potentially until the walls that define recesses 110a, 110b, 112a, 112b touch) to provide a narrow width configuration, or outwardly away from one another to provide a wide width configuration, from the position shown in FIG. 10. That enables the tray to accommodate teeth of an upper jaw that are asymmetric or to accommodate arches having different sizes.

The hinges 108a, 108b are “live” hinges that are formed by grooves which leave sections of the tray with reduced wall thickness. The trays will be formed from a suitable thermoplastic polymeric material such as polypropylene for example. The selected material will be able to withstand high deformations while still being able to return to its original position. Polypropylene, for example, has a molecular structure with long chains of molecules which are loosely bonded to each other, which allows the chains to move among themselves without tearing the bonds that hold the chains together.

The anterior portion 103 of the tray defined by walls 107a, 111a, and base wall 109a is relatively rigid, as are each of the posterior portions 105a, 105b of the tray rearward of the hinges 108a, 108b. The hinges form relatively flexible regions between the relatively rigid anterior portion 103 and posterior portions 105a, 105b. The relatively rigid anterior and posterior portions provide a secure mounting of the tray to the subject's teeth.

Rather than having hinges that are incorporated into the moulding, the flexible regions could be defined by pivots or other types of hinges for example.

The upper tray is configured to receive a suitable substance to form a mould around the teeth of the subject's upper dentition, to mount the upper tray to the subject's teeth and thereby enhance the attachment to the tray to the teeth. The substance would typically be a suitable type of dental putty for example, which will be inserted into the tray prior to the tray being installed onto the user's upper dentition. The putty will set around the user's teeth, and will couple the tray to the user's teeth.

Retention features 113 are provided in the base walls 109a, 109b, 109c of the upper tray to enhance bonding between the dental putty and the surfaces of the tray, to minimise movement therebetween once the putty sets. In the form shown, the retention features are apertures. The features could be any suitable type of features, such as ribs, slots, raised features, or dimples for example. Retention features may also be provided in the walls 107a, 107b, 107c, 111a, 111b, 111c of the tray.

The dental putty can be any suitable substance. In one embodiment, the putty is an elastomeric substance such as an “addition-cured” silicone or polyether silicone for example.

The upper tray is also provided with a forwardly-projecting lip 115 that projects from the anterior portion 103 of the tray. The lip can be used as a handle to facilitate insertion, alignment, removal of the tray and also acts to support the soft tissues of the upper lip in part.

The lower tray 201 is shown separated from the upper tray in FIGS. 16 to 22. Unless described below, the lower tray should be considered to have the same features as the upper tray, and like numerals are used to indicate like parts with the addition of 100. The lower tray will be installed on the lower dentition in the same manner discussed above.

As can be seen in FIG. 16, the bases of the posterior portions of lower tray differ from the upper tray. In the form shown, the bases of the posterior portions have a plurality of transverse ribs 215 of varying lengths that project downwardly from the base walls 209b, 290c toward the region of the tray that receives the subject's dentition. The ribs toward the rear ends of the posterior portions are shallower than the ribs at the forward ends of the posterior portions. The result is that the posterior portions have angled surfaces that form forwardly inclined ramps 217 that become progressively deeper from the rear of the posterior portions toward the front of the posterior portions. That is, the heights of the ramps toward the rear of the tray are less than the heights of the ramps toward the front of the tray, forming an incline from a posterior to an anterior direction in the lower tray. The bases of the ramps are indicated by broken lines in FIG. 16.

In the embodiment shown, the ramps become progressively deeper from the rear of the posterior portions of the tray, right to the forward edges of the ramps, and the forward edges of the ramps terminate in generally vertical walls to form shoulders as described below.

As well as forming the ramps, the ribs act as retention features to enhance bonding between the dental putty and the tray, to minimise movement therebetween once the putty sets. The lower tray may also have further retention features, such as those described in relation to the upper tray, to enhance bonding between the dental putty and the tray.

The ribs may be integrally formed with the ramps. Alternatively, the ramps could be hollow, with the ribs separately formed and fastened to the underside of the ramps.

The ramps act as spacers to maintain a spacing between at least the anterior portions 103, 203 of the upper and lower trays when installed on the upper and lower dentitions of the subject. The ramps enable the subject's rear teeth to be closer together than the subject's front teeth, with the spacing between the subject's front teeth being sufficient for the tube 401 to extend therebetween.

Each ramp has an arcuate surface that faces the upper tray in use. The arcuate surface has a convex curvature when viewed from the upper tray, and is curved in a forward/rearward direction of the device. The ramps move with the lower jaw as the lower jaw is moved forward relative to the upper jaw. The curvature of the ramps is designed to accommodate the rotation and translation of the condyle in the glenoid fossa when the mandible is protruded, to mimic anatomical function.

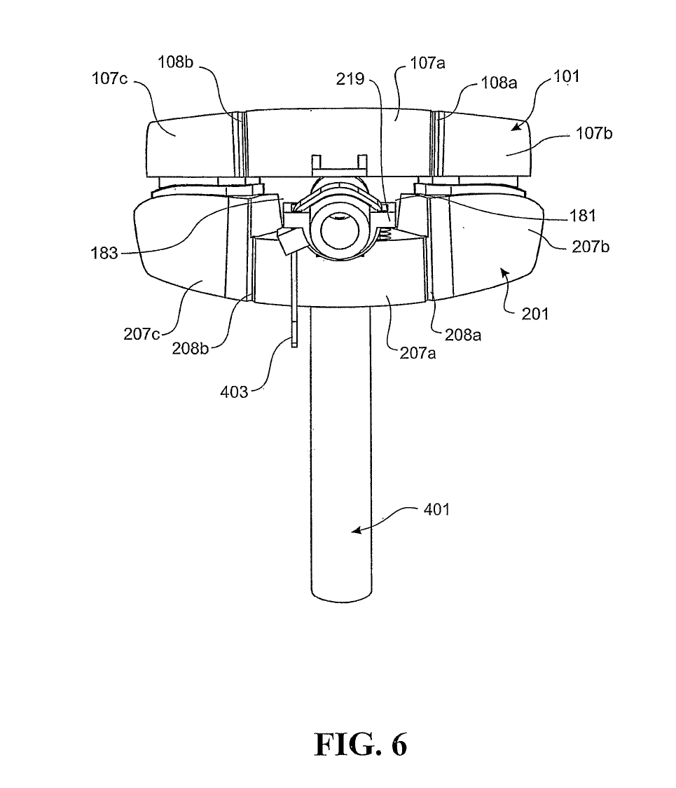

Each ramp 217 terminates near, and in the embodiment shown at, the anterior portion of the lower tray to form a shoulder or step between the respective posterior portion and the anterior portion of the tray. In the embodiment shown, the shoulders are positioned at the edge of the recesses 210a, 210b that provide the flexible regions of the trays. The shoulders extend upwardly above the base 209a of the anterior portion of the lower tray. The shoulders form an enlarged spacing or opening in the anterior portion of the tray, as shown in FIG. 6. The anterior opening is designed to be of sufficient size to allow an endotracheal tube to extend through the opening to enable intubation of the subject to occur. That is, the shoulders are at least as high as a diameter of the endotracheal tube.

For adult use, an endotracheal tube may typically have an outer diameter of between 8.7 mm and 12.7 mm. Accordingly, if the device is configured for adult use, the heights of the shoulders of the ramps may be at least about 9 mm, in another embodiment at least about 10 mm, in another embodiment at least about 11 mm, in another embodiment at least about 12 mm, and in another embodiment at least about 13 mm. If smaller endotracheal tubes are to be used, the heights of the shoulders could be smaller. If larger endotracheal tubes are to be used, the heights of the shoulders could be larger. The heights of the shoulders will advantageously be selected so that one device is able to be used with several different sizes of endotracheal tubes.

The opening may be sufficiently sized that it provides side openings 181, 183 on either side of the endotracheal tube. The side openings 181, 183 enable a subject wearing the device to have fluids removed by suction, if necessary.

Additionally, the anterior opening facilitates mouth breathing when intubation is not required but when the nasal passages are compromised, and would allow passive exit of fluid secretions. The ramps also provide a vertical occlusal stop that prevents biting down on the endotracheal tube.

In one embodiment, the vertical spacing between the anterior portions of the upper and lower tray is about 13 mm, at its widest point the width of the anterior opening is about 38 mm, and at its narrowest point the width of the anterior opening is about 24 mm.

In the embodiment shown, the ramps extend from substantially the rear of the tray and terminate at or near the anterior portion of the tray. By providing elongate ramps that are forwardly inclined and that terminate at or near the anterior portion of the tray, the upper and lower dentitions are supported in a natural position to minimise strain on the subject's facial muscles and dentitions during use of the device. The elongate ramps also minimise pressure points on the patient's teeth or gums by increasing the surface of the upper and lower trays (or between the lower tray and the upper dentition if only the lower tray is used).

By having the ramps terminating at or near the anterior portion of the tray, and of sufficient height that an intubation tube can be inserted through the opening between the ramps, a subject wearing either the lower or both trays will not be able to bite down on the intubation tube.

Additionally, by having ramps with that configuration, the ramps minimise any potential for forward tipping of the unstable maxilla in a subject with a severe mid-face injury (as in a complete Le Fort I or II mid-face injury for example).

The spacers may be provided on one or both of the upper and lower trays. It will be appreciated that both of the upper and lower trays could be provided with ramps having approximately half the height of that shown for the lower tray, so that a similar spacing to that shown is provided between the anterior portions of the trays. However, if the ramps are provided on the lower tray, and are each provided with a sufficient height that they can prevent a subject from biting down on an intubation tube, the lower tray can be used alone for stabilising an intubation tube relative to the lower jaw.

In an alternative embodiment, the spacers need not be in the form of ramps. By way of example only, the spacers could be configured such that the surfaces of the spacers distal the dentition that the tray is to be mounted on, are generally parallel to that dentition in use. However, the forwardly inclined ramps provide the functional benefits described above.

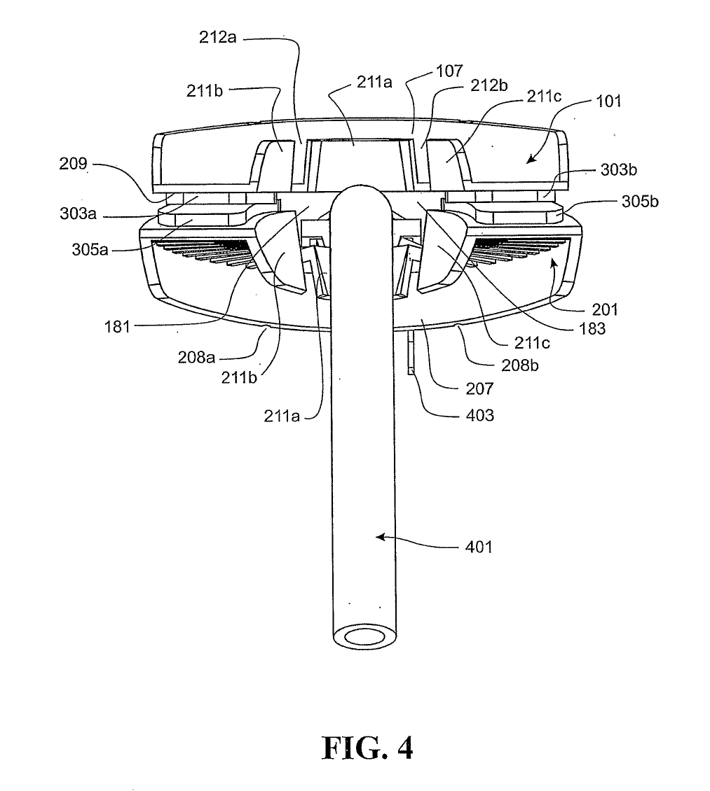

The device further comprises engagement features 301 to engage the posterior portions of the upper and lower trays 101, 201 to one another, and that minimise lateral movement of the trays relative to one another once the trays are engaged with one another. As shown in FIGS. 4 and 27 for example, the engagement features 301 are provided to directly engage the posterior portions of the trays to each other. The engagement features may be provided on one or both of the upper and lower trays.

In the embodiment shown, the left side engagement feature that engages the left posterior portions 105a, 205a of the upper and lower trays comprises a first portion 303a mounted to the underside of the base wall 109b of the left posterior portion 105a of the upper tray, and a second portion 305a mounted to the upper side of the base wall 209b of the left posterior portion 205a of the lower tray. That is, the left side engagement feature portions are provided on facing contact surfaces of the left posterior portions of the upper and lower trays. Similarly, the right side engagement feature comprises a first portion 303b that is mounted to the underside of the base wall 109c of the right posterior portion 105b of the upper tray, and a second portion 305b that is mounted to the upper side of the base wall 209c of the right posterior portion 205b of the lower tray. That is, the right side engagement features are provided on facing contact surfaces of the right posterior portions of the upper and lower trays. By positioning the engagement feature portions on facing contact surfaces of the trays, the engagement features can directly engage with one another to stabilise the upper and lower tray relative to one another, and thereby stabilise the subject's upper and lower jaw relative to one another.

By positioning the engagement features, and the spacers, on the posterior portions of the trays, the engagement features and spacers do not interfere with the spacing that is provided between the anterior portions of the upper and lower trays and thereby between the subject's upper and lower front teeth.

In one embodiment, the engagement features are provided on upper surfaces of the ramps 217 that maintain the spacing between the subject's front teeth, and on undersides of the posterior portions of the upper tray. By positioning the engagement features on the upper surfaces of the ramps of the lower tray, and by having the ramps extend from substantially the rear of the tray and terminate near, and in the embodiment shown at, the anterior portion of the tray, a large number of possible forward/rearward engagement positions of the trays are provided.

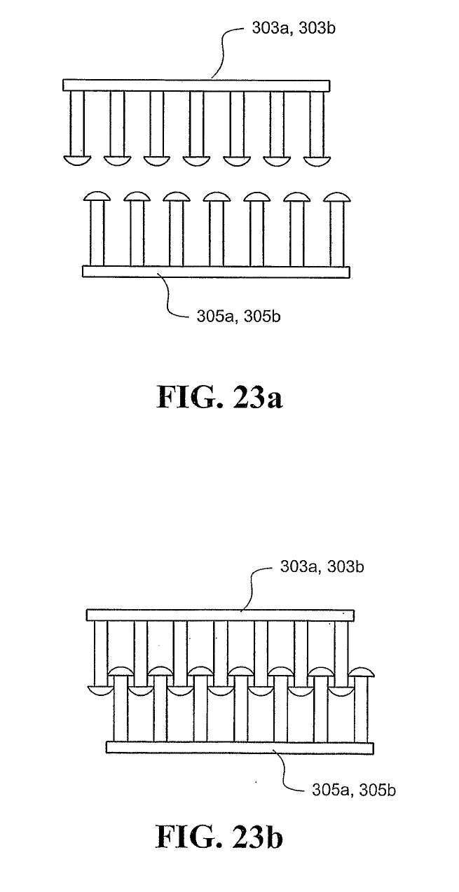

In the form shown in FIGS. 23a, 23b, the engagement features 301 comprise opposed strips 303a, 303b, 305a, 305b having a plurality of mushroom-shaped protrusions, with the mushroom-shaped protrusions being engageable with one another. A suitable product is marketed under the trade mark DUAL LOCK by 3M. The backing surfaces that carry the mushroom-shaped protrusions will generally be self-adhesive, so they can be adhered to the surfaces of the trays. The trays may be provided with the engagement features pre-adhered, or that could be done in the field if need be. The engagement features may be moulded into the upper and lower trays during manufacture of the trays. The engagement features may be robotically inserted into the mould, prior to injection of the polymeric resin.

The engagement features 301 provide a plurality of possible forward-to-rearward engagement positions of the trays, and once engaged, minimise forward-to-rearward movement of the trays relative to one another. Once engaged, the engagement features may in one embodiment substantially prevent, and may in another embodiment prevent, forward-to-rearward movement of the trays relative to one another.

Additionally, the engagement features 301 minimise lateral movement of the trays relative to one another once the trays are engaged with one another. The engagement features provide a plurality of possible lateral or side-to-side engagement positions of the trays, and once engaged, minimise side-to-side movement of the trays relative to one another. Once engaged, the engagement features may in one embodiment substantially prevent, and may in another embodiment prevent, side-to-side movement of the trays relative to one another.

The engagement feature(s) enable little or no side-to-side or forward-to-rearward movement of the trays once engaged. The amount of any possible movement is significantly less than the movement that would otherwise be possible in the absence of the engagement features.

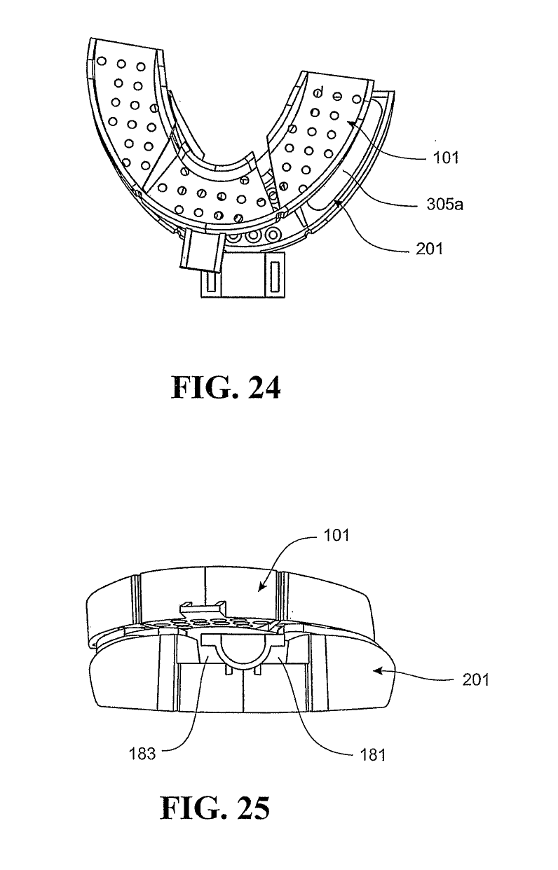

As well as providing a plurality of forward-to-rearward and side-to-side engagement positions of the trays 101, 201, the engagement features also accommodate angular misalignment of the trays as shown in FIGS. 24 and 25. That is, it is not necessary for the upper and lower trays to be perfectly aligned for the trays to be engaged to one another. That is beneficial for use with trauma subjects, as the subject's upper and lower jaws do not need to be forced into a specific position to engage the trays to one another. Generally, in the case of jaw fractures, the subject's jaws could not be forced into a properly aligned position without causing additional pain or damage. The forward-to-rearward engagement positions also enable the trays to be used to cause mandibular advancement (forward movement of the lower jaw relative to the upper jaw). The mandibular advancement can protect or maintain airway patency in a subject that has the device installed.

In the various relative angular positions of the upper and lower trays, the engagement features can be engaged to engage the trays to one another, and the engagement features will minimise lateral and forward/rearward movement of the trays once engaged.

In the case of a subject who has a fracture that has separated the upper jaw from the skull, the forward-to-rearward engagement positions enable the upper jaw of the subject to be reduced into normal anatomical relationship and stabilised in a desired position relative to the lower jaw thereby reducing pain, potentially life-threatening bleeding (maxillary fractures are extremely bloody and difficult to manage) and airway obstruction.

The engagement features are disengageable so that the upper and lower trays can be manually moved apart if necessary, such as to remove the device from a subject's mouth after use. In the form shown, the engagement features attach the upper and lower trays to one another. That is, the engagement features will engage with sufficient strength that the upper and lower trays will not inadvertently separate once engaged and installed onto a subject's upper and lower dentitions. However, alternative engagement features could be used, such as those described below, and the alternative engagement features may just engage the trays to one another rather than causing physical attachment of the trays to one another. Such a configuration may make the trays easier to remove from the subject after use, or easier for the subject's jaws to be repositioned in use.

Once the posterior portions of the trays are disengaged, or prior to engagement of the posterior portions of the trays, the device does not interfere with the subject's normal lateral and forward/rearward movement of the jaws relative to one another.

Rather than using engagement features with mushroom-shaped protrusions as outlined above, the engagement features may each be of any suitable type. For example, the engagement features may comprise at least one member on one of the trays that is engageable with at least one member on the other of the trays. In the form shown, the engagement features each comprise a plurality of members, and the members on one of the trays comprise enlarged heads that engage with enlarged heads of the members on the other of the trays. The member(s) on one tray may comprise projection(s), and the members on the other tray may comprise recess(es). Alternatively, each tray may comprise both projection(s) and recess(es). As another example, one or more rigid projections may extend from one tray and engage with corresponding projection(s) on the other tray. In one possible form, the engagement features may each comprise a projection on one of the trays that is selectively engageable with a plurality of recesses on the other of the trays.

As another example, the engagement features may comprise a hook and loop fastening system, where a plurality of hooks on one of the trays engage with a plurality of loops on the other of the trays. A suitable fastening system is marketed under the trade mark VELCRO. The engagement feature(s) may be integrally formed with the trays, or may be attachable thereto or moulded with the trays.

In an alternative configuration, the engagement features may comprise adhesive surfaces that engage facing surfaces of the posterior portions of the trays. The adhesive surfaces may be provided on only one of the trays (such as on the upper sides of the ramps for example), and may adhere to the undersides of the posterior portions of the upper tray. The configuration could be reversed, with adhesive surfaces provided on the underside of the posterior portions of the upper tray, and no adhesive surfaces provided on the upper sides of the lower tray. Alternatively, on one side of the device the adhesive surface may be on an upper side of the ramp, and on the other side of the device the adhesive surface may be on an underside of the posterior portion of the upper tray. Alternatively, adhesive surfaces may be provided on the upper sides of the ramps of the lower tray and on the undersides of the posterior portions of the upper tray. The adhesive surfaces provide the same functionality and benefits as the other engagement features described above.

The lower tray is also provided with a locator 219 for receipt of an endotracheal tube 401 and that minimises or prevents unwanted movement of the endotracheal tube 401 once inserted in use. The locator is in the form of a tongue that is substantially aligned with the opening in the anterior portion of the tray, and extends from an external anterior portion of the tray as shown so that the locator can be readily accessed from the front of the subject's mouth. The locator 219 is an arcuate shape, and acts as a locator for receiving part of the endotracheal tube 401, to assist with maintaining the tube in a desired position in use. As the locator projects from the anterior portion of the tray, the locator can also be used as a handle to facilitate insertion, alignment, and removal of the tray.

As shown, the locator comprises a recess that faces the upper tray when the trays are mounted to the subject's upper and lower dentitions. The recess has an upwardly-open concave receiving surface for receiving and supporting part of an endotracheal tube with a curved outer periphery.

The concave receiving surface may comprise a frictional, textured, or adhesive surface for minimising movement of an endotracheal tube when received in the locator.

A fastener-receiving region 221a, 221b is configured for receipt of a fastener 403 for fastening the endotracheal tube 401 to the locator, to minimise or prevent unwanted movement of the endotracheal tube once inserted. The fastener-receiving region is provided on an external anterior portion of the lower tray, so the fastener-receiving region can be readily accessed from the front of a subject's mouth. In the form shown, the fastener-receiving region comprises a support formed on either side of the arcuate recess of the locator, and comprises two apertures 221a, 221b, one on either side of the recess. The two apertures 221a, 221b are configured for receipt of any suitable type of fastener, such as a tie or strap that can partly surround the tube for example. The support may be suitable for receipt of a cable tie-type strap 403 as shown.

As shown in FIG. 1, a suitable fastener 403 Can extend around the base of the recess and through the apertures 221a, 221b, to surround the upper portion of the endotracheal tube 401. The fastener can then be pulled tight, to minimise or prevent unwanted movement of an endotracheal tube once inserted. The fastener 403 may be a cable tie-type tie or strap, that has a ratchet mechanism that enables the device to be tightened easily, but requires the device to be cut for removal. However, as there is benefit in having the device readily removable, rather than using a cable tie-type tie, the fastener may be readily removable.

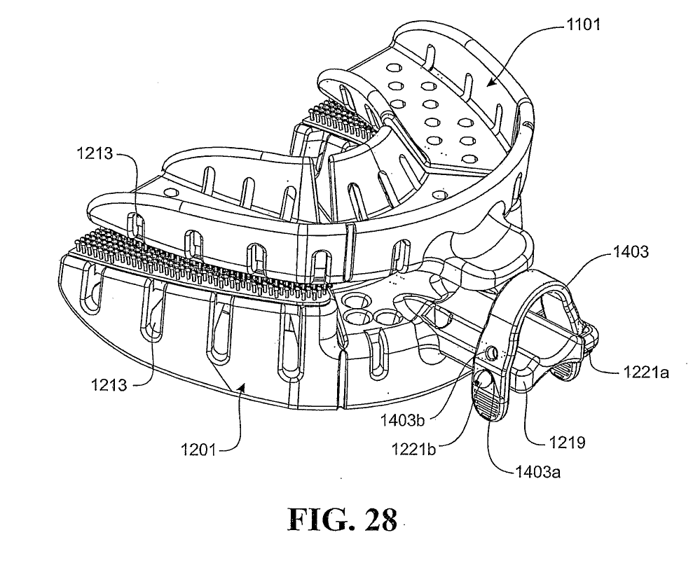

FIG. 28 shows a second embodiment device. Unless described below, the features, functionality, and method of its use will be as described herein for the first embodiment of FIGS. 1 to 27, and like reference numerals indicate like parts with the addition of 1000.

In the embodiment of FIG. 28, the fastener 1403 and fastener support differ from the device described above. Again, the fastener 1403 is a strap. However, the fastener 1403 is readily disengageable from the fastener support to enable removal of the endotracheal tube (not shown). One end of the fastener 1403 is retained in recess 1221a of the fastener support. The opposite side of fastener support has a bulbous projection 1221b. The other end of the fastener is engageable with the bulbous projection 1221b by inserting the bulbous projection through an aperture 1403a, 1403b of the fastener. The fastener has a plurality of apertures, to provide adjustment of the length of the fastener. That enables the device to support different sized endotracheal tubes.

The slots 1213 that are visible in the sides of the device act as retention features to enhance adhesion of the putty to the device.

Reverting to the first embodiment, the locator 219 and the fastener 403 hold the endotracheal tube 401 in a desired position with a forward part 401a of the endotracheal tube projecting from the front of the trays a sufficient distance that it will clear the subject's lips, and a rearward part 401b of the endotracheal tube extending a sufficient distance down the subject's airway to ensure adequate ventilation of the lungs via the endotracheal tube. The corresponding features of the embodiment of FIG. 28 will function in the same way.

The device may be provided in kit form with at least one upper tray 101, at least one lower tray 201, and at least one fastener 403. When the trays are provided with flexible regions so that the trays can be manipulated to accommodate dental arches of different sizes or shapes, a single upper tray and a single lower tray may be provided in the kit. However, if trays are used that do not have flexibility, the kit may comprise a plurality of upper and lower trays of different sizes, to accommodate different dental arch sizes. The different sized trays may be colour coded.

The kit will also include the engagement features which may initially be separate from the trays and adapted to be positioned on the posterior portions of the tray(s), or may be attached to or formed as part of the tray(s) in the kit.

The kit may also be provided with one or more spare fasteners 403, in case the strap is damaged in use.

The kit may also Comprise a suitable substance applicator for applying substance that is used when mounting the trays to a subject's teeth, such as dental putty for example.

The kit may further comprise instructions for use.

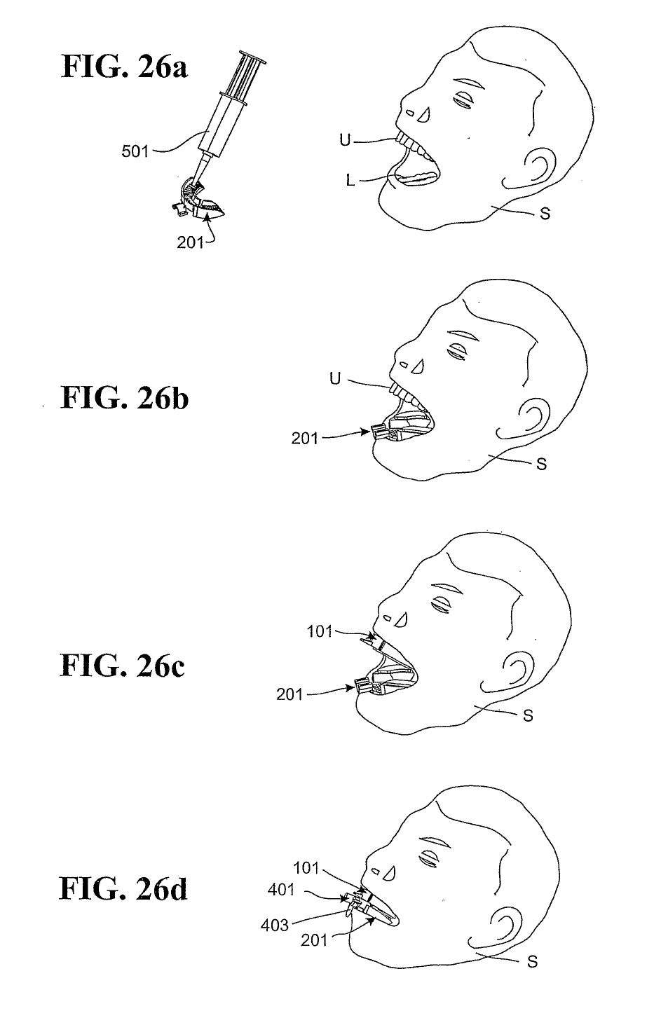

FIGS. 26a to 26d schematically show a method of using either of the devices described above to stabilise a jaw of a subject S having an upper dentition U and a lower dentition L. Initially, a substance such as dental putty is applied from applicator 501 into one of the trays, such as lower tray 201 for example. After application of the substance, the lower tray is installed onto the subject's lower dentition L as shown in FIG. 26b. The substance is then applied from the applicator 501 into the other of the trays, and in this case into upper tray 101. After application of the substance, the upper tray 101 is mounted to the user's upper dentition U as shown in FIG. 26c. It will be appreciated that the upper and lower trays can be installed in either order or concurrently.

When required, the endotracheal tube 401 is inserted between the anterior portions of the upper and lower trays, and down the subject's throat. The tube is mounted to the lower tray 201 and fastened by the fastener 403, so that part of the tube extends from the user's mouth.

The endotracheal tube can be inserted and mounted either before or after the trays are engaged to one another in the position shown in FIG. 26d.

The endotracheal tube may be inserted down the subject's throat prior to installing the upper and lower trays. In that embodiment, part of the endotracheal tube will be positioned between the anterior portions of the upper and lower trays during installation of the upper and lower trays. Alternatively, the endotracheal tube may be inserted down the subject's throat, and part of the endotracheal tube positioned between the anterior portions of the upper and lower trays, after installation of the upper and lower trays. In that embodiment, part of the endotracheal tube may be positioned in the opening at the anterior portion of the lower tray concurrently with the insertion of the endotracheal tube down the subject's throat or after the insertion of the endotracheal tube down the subject's throat.

The trays are engaged to one another by moving the posterior portions of the trays toward one another so the engagement features engage the posterior portions of the trays to each other. Once engaged, lateral movement of the trays relative to one another is minimised, and a spacing is maintained between the anterior portions of the trays.

If required, an operator can grip the tube locator 219 to pull the lower tray 201 and therefore the user's lower jaw forwardly relative to the upper jaw, before the trays are engaged to one another. Once the trays are engaged to one another, the engagement features minimise lateral movement between the trays, and in one embodiment substantially prevent lateral movement between the trays.

The width of the upper and lower trays can be adjusted to fit to the dentitions of the subject.

When the upper and lower jaws are angularly misaligned, the method may comprise engaging the engagement feature(s) without correcting the angular misalignment.

Once the subject has been transported to a medical facility, the process can be reversed by removing the trays from user's upper and lower dentitions, and extracting the tube from the user's airway if a tube has been used. However, removal of the trays is not essential, as the configuration of the device is such that the device can remain installed on a subject during surgery, as an anaesthetist can deliver anaesthetic to the subject via the endotracheal tube 401. The device will also provide a stable facial platform for anaesthetists, ICU and/or ED staff or medics when bag masking the subject, as the bag mask can be applied over the patient's face without interference from the device.

If necessary, after the device has been removed, it can be reinstalled if required. That may or may not require the addition of new substance to the interiors of the trays.

The device stabilises the upper jaw relative to the lower jaw, and if necessary advances the lower jaw relative to the upper jaw to achieve mandibular advancement. As the trays each have a relatively rigid anterior portion that substantially fully surrounds the subject's front teeth and relatively rigid posterior portions that substantially fully surround the subject's posterior teeth, in the case of a fractured jaw, once the trays are installed onto the subjects upper or lower dentition, the trays will stabilise the fracture segments of the respective jaw to minimise rubbing of bone ends of the fracture segments. This will minimise pain, bleeding, and will control secretions. When adjacent bone ends of the fracture segments are misaligned, the method may comprise, prior to installing the tray onto the subject's dentition, generally aligning the bone ends. When adjacent bone ends of the fracture segments are aligned or generally aligned, the tray can be installed onto the subject's dentition without moving the fracture segments.

The lower tray can be used either alone or in combination with the upper tray for fracture reduction. Similarly, the upper tray can be used alone or in combination with the lower tray for fracture reduction.

In the case of a fractured jaw, the device may achieve complete alignment of the adjacent bone ends of the fracture segments, and therefore anatomical reduction or return to normal anatomy. However, it will be appreciated that some offset in the bone ends will still provide satisfactory results. Therefore, in the case of a fractured jaw, the device may achieve anatomical reduction within variations of the clinical situation.

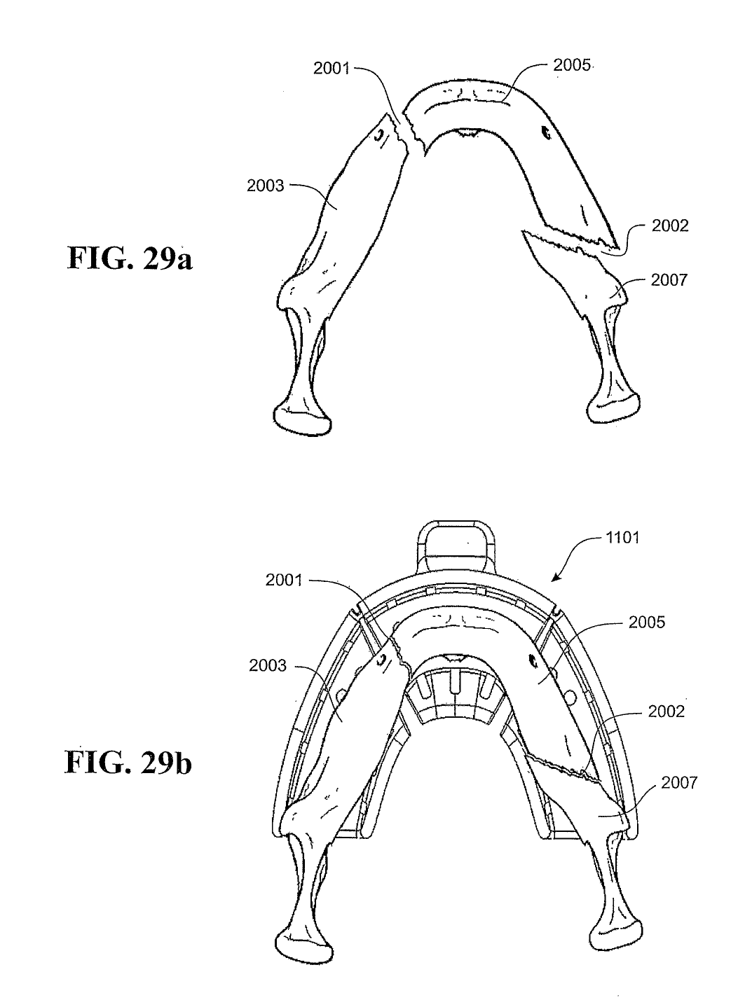

FIG. 29a shows a fractured jaw that can be stabilised with the device of FIG. 28 (or alternatively with the device of the earlier figures). The jaw may be an upper jaw or lower jaw. The jaw has two fractures 2001, 2002, which form three fracture segments 2003, 2005, 2007. Each fracture segment has a bone end adjacent the fracture 2001, 2002. It can be seen that the bone ends in the fractured jaws are misaligned.

FIG. 29b shows the fractured jaw after the application of the tray 1101. While not shown in FIG. 29b, it will be appreciated that dental putty will be provided in the tray. The adjacent bone ends have been substantially aligned as the tray has been applied to the fractured jaw.

In an alternative embodiment, the device may comprise a lower tray of the type described for either of the first and second embodiments above, and will be suitable for us in stabilising an endotracheal tube relative to the jaw, without requiring the tube to be externally secured to the subject's face or neck or inserted via a surgical airway in the subject's neck. The lower tray could have any one or more of the features outlined in relation to the embodiments described above, and will have at least the forwardly inclined ramps with shoulders defining an opening at the anterior portion of the tray that is of a sufficient size to enable an endotracheal tube to extend therethrough, and a locator for receiving a portion of an endotracheal tube. The upper tray need not be provided, nor would the engagement features that engage the trays to one another. The lower tray and endotracheal tube can be installed using the same method described above for the two-tray embodiment devices.

Again, the device could be provided in kit form comprising the lower tray and a substance for use in mounting the tray to a subject's dentition. The kit may also comprise an applicator for the substance, a plurality of fasteners, a plurality of trays of different sizes (if the lower tray is not provided with the flexible regions), and instructions for use.

At least some embodiments of the present invention provide an intra-oral stabilisation device that can be used to temporarily stabilise a jaw in a non-invasive manner (without the use of sharps). At least some embodiments can be easily and rapidly inserted into a subject's mouth and removed after use. The nature of at least some embodiments is such that the devices are able to provide the desired functionality without worsening any existing tissue or bone damage. Two tray devices in accordance with at least some embodiments of the present invention provide lateral stabilising of the upper jaw relative to the lower jaw, as well as the ability to stabilise fracture segments of one or both jaws. Two tray devices in accordance with at least some embodiments of the present invention can be used to advance and stabilise the mandible in a semi-conscious or unconscious subject, and can stabilise the mandible in a normal anatomic position in a conscious subject. Single tray devices in accordance with at least some embodiments of the present invention can also be used to stabilise fracture segments of fractured upper or lower jaws. The devices in accordance with at least some embodiments are reinsertible into the same subject, readily removable, and can be used to reposition the jaw to a normal or substantially normal anatomy in the case of jaw fracture(s). Devices in accordance with at least some embodiments also provide airway management for the subject, by enabling positioning and stabilisation of the jaws relative to one another, and/or by stabilising an intubation tube relative to the lower jaw and preventing a subject from biting down on the tube. In the case of subjects who are not intubated, the anterior spacing in at least some embodiments will be sufficient to enable the subject to expel fluid or vomit.

The devices may be disposable or may be re-used in the same patient within a 24-hour period. After that period the impression of the dentition in the dental adhesive may have changed dimensionally due to warping or creep and bacterial contamination may pose an infection risk to the subject.

The devices and methods have been described with reference to subjects who have suffered trauma, who may have fractured jaws, and who may require airway management. However, the devices also have other applications. For example, the devices could be used in initial trauma management by way of jaw positioning to maintain airway patency in an unconscious subject at roadside or in the field; for bag-mask ventilation; for minimising or preventing trauma to the teeth from grinding and damage to the tongue from biting or chewing in closed head injury subjects; in intensive care units as an adjunct in airway management, for dental implant surgery planning as a measuring or locating device with radiographic markers, or as an aid for orthognathic surgical planning allowing the mandible to be positioned anteriorly to help gauge the amount of movement gained from surgical repositioning of the jaws.

The above describes embodiments of the present invention only, and modifications can be made thereto without departing from the scope of the present invention.

For example, not all of the features described herein need to be provided in every device. Devices in accordance with alternative embodiments of the present invention may have only some of the features described herein. Therefore, if should be appreciated that not every advantage described above needs to be met by every embodiment of the present invention.

As another example, the devices are configured for use with a human subject. It will be appreciated that the shape could be modified to be suitable for use with a non-human mammal, and particularly for simians and primates.

Other example modifications are outlined in the description above, including in the “Summary of the Invention” section.

1.-63. (canceled)

64. A device for stabilising a jaw relative to the other jaw, the device comprising:
an upper tray for installing onto the upper dentition of a subject, the upper tray having an anterior portion and two posterior portions;
a lower tray for installing onto the lower dentition of the subject, the lower tray having an anterior portion and two posterior portions;
at least two spacers to provide a spacing between the anterior portions of the upper and lower trays when installed on the upper and lower dentitions of the subject, wherein at least one spacer is provided between corresponding posterior portions on one side of the upper and lower trays and at least one other spacer is provided between corresponding posterior portions on the other side of the upper and lower trays, and wherein said at least one spacer comprises a forwardly inclined ramp having a forwardly inclined upper side and positioned on a left posterior portion of the lower tray and said at least one other spacer comprises a forwardly inclined ramp having a forwardly inclined upper side and positioned on a right posterior portion of the lower tray; and
engagement features for engaging undersides of the posterior portions of the upper tray with the forwardly inclined upper sides of the forwardly inclined ramps of the posterior portions of the lower tray, and that minimise lateral movement of the trays relative to one another once the posterior portions of the trays are engaged with one another.

65. A device as claimed in claim 64, wherein the engagement features provide a plurality of possible forward-to-rearward engagement positions of the trays, and once engaged, minimise forward-to-rearward movement of the trays relative to one another.

66. A device as claimed in claim 65, wherein once engaged, the engagement features substantially prevent forward-to-rearward movement of the trays relative to one another.

67. A device as claimed claim 64, wherein the engagement features provide a plurality of possible side-to-side engagement positions of the trays, and once engaged, minimise side-to-side or lateral movement of the trays relative to one another.

68. A device as claimed in claim 62, wherein once engaged, the engagement features substantially prevent side-to-side movement of the trays relative to one another.

69. A device as claimed in claim 64, wherein the engagement features enable the trays to be engaged to one another, and for lateral movement between the trays to be minimised or substantially prevented, in a plurality of relative angular positions of the trays.

70. A device as claimed in claim 64, wherein an engagement feature is provided on each of the posterior portions of the upper tray, and a further engagement feature is provided on each of the forwardly inclined upper sides of the forwardly inclined ramps of the posterior portions of the lower tray, to engage the posterior portions of the trays with one another.

71. A device as claimed in claim 70, wherein the engagement features comprise a plurality of members on each of the posterior portions of the upper tray, and the members are engageable with a plurality of members on each of the forwardly inclined upper sides of the forwardly inclined ramps of the posterior portions of the lower tray.

72. A device as claimed in claim 71, wherein the members on the upper tray comprise enlarged heads that engage with enlarged heads of the members on the lower tray.

73. A device as claimed in claim 71, wherein the engagement features comprise hook and loop fasteners.

74. A device as claimed in claim 64, wherein on a left posterior portion of the device, an engagement feature is provided on one of the underside of the upper tray and the forwardly inclined upper side of the left forwardly inclined ramp of the lower tray, and on a right posterior portion of the device, an engagement feature is provided on one of the underside of the upper tray and the forwardly inclined upper side of the right forwardly inclined ramp of the lower tray.

75. A device as claimed in claim 64, wherein the engagement features are disengageable so the upper and lower trays can be manually moved apart if necessary, but the engagement features will engage with sufficient strength that the upper and lower trays will not inadvertently separate when engaged and the trays are installed on a subject's upper and lower dentition.

76. A device as claimed in claim 64, wherein the spacers provide a sufficient spacing between the anterior portions of the upper and lower trays when installed on the upper and lower dentitions of the subject that an endotracheal tube can be inserted between the anterior portions of the trays.

77. A device as claimed in claim 64, wherein the ramps comprise ribs of varying heights.

78. A device as claimed in claim 64, wherein each ramp has an arcuate surface that faces the upper tray in use, that is convex when viewed from the upper tray, and that is curved in a forward/rearward direction of the device.

79. A device as claimed in claim 64, wherein the ramps each terminate at or near the anterior portion of the lower tray to form a shoulder, and the shoulders form a spacing in the anterior portion of the lower tray that is sufficient to receive part of an endotracheal tube.

80. A device as claimed in claim 64, wherein the engagement features are provided on the ramps.

81. A device as claimed in claim 64, wherein the lower tray comprises a locator for receipt of part of an endotracheal tube, to assist with maintaining the tube in a desired position in use.

82. A device as claimed in claim 81, comprising a fastener-receiving region that is configured for receipt of a fastener for fastening the endotracheal tube to the locator, to minimise or prevent unwanted movement of the endotracheal tube once inserted.

83. A device as claimed in claim 82, further comprising a fastener.

84. A device as claimed in claim 64, wherein at least one of the trays comprises at least one flexible region, to enable the tray to be mounted to an asymmetric dental arch or to dental arches of different sizes.

85. A device as claimed in claim 84, wherein the tray(s) comprising the flexible region(s) further comprises a relatively rigid anterior portion for supporting a subject's front teeth, two relatively rigid posterior portions for supporting a subject's side and posterior teeth on respective sides of the dental arch, and a flexible region between the anterior portion and each of the posterior portions.

86. A device as claimed in claim 85, wherein the lower tray comprises the flexible regions, with each ramp terminating at a respective one of the flexible regions to form respective shoulders, and with the shoulders forming a spacing in the anterior portion of the lower tray that is sufficient to receive part of an endotracheal tube.

87. A device as claimed in claim 84, wherein the engagement features are configured to accommodate a plurality of relative adjusted widths of the posterior portions of the trays, but to engage the trays to one another and minimise lateral movement of the trays once engaged.

88. A device for stabilising a jaw relative to the other jaw, the device comprising:
an upper tray for installing onto the upper dentition of a subject, the upper tray having an anterior portion and two posterior portions;
a lower tray for installing onto the lower dentition of the subject, the lower tray having an anterior portion and two posterior portions;
at least two spacers to provide a spacing between the anterior portions of the upper and lower trays when installed on the upper and lower dentitions of the subject, wherein at least one spacer is provided between corresponding posterior portions on one side of the upper and lower trays and at least one other spacer is provided between corresponding posterior portions on the other side of the upper and lower trays, and wherein said at least one spacer comprises a forwardly inclined ramp on a left posterior portion of the lower tray and said at least one other spacer comprises a forwardly inclined ramp on a right posterior portion of the lower tray, and wherein each ramp has an arcuate surface that faces the upper tray in use, that is convex when viewed from the upper tray, and that is curved in a forward/rearward direction of the device; and
engagement features for engaging undersides of the posterior portions of the upper tray with upper sides of the posterior portions of the lower tray, and that minimise lateral movement of the trays relative to one another once the posterior portions of the trays are engaged with one another.

89. A kit of parts for a device as claimed in claim 64, the kit comprising:
an upper tray for installing onto the upper dentition of a subject, the upper tray having an anterior portion and two posterior portions;
a lower tray for installing onto the lower dentition of the subject, the lower tray having an anterior portion and two posterior portions;
at least two spacers to provide a spacing between the anterior portions of the upper and lower trays when installed on the upper and lower dentitions of the subject, wherein the spacers comprise forwardly inclined ramps having forwardly inclined upper sides, and wherein a forwardly inclined ramp is provided on each posterior portion of the lower tray; and
engagement features for engaging undersides of the posterior portions of the upper tray with the forwardly inclined upper sides of the forwardly inclined ramps of the posterior portions of the lower tray, and that are positioned on or adapted to be positioned on one or both of the trays.

90. A method of stabilising a subject's jaw relative to their other jaw, comprising:
providing a device as claimed in claim 64;
installing the upper tray onto the subject's upper dentition;
installing the lower tray onto the subject's lower dentition; and
engaging the posterior portions of the upper and lower trays to one another by engaging the undersides of the posterior portions of the upper tray with the forwardly inclined upper sides of the forwardly inclined ramps of the lower tray, to minimise lateral movement of the trays relative to one another and to maintain a spacing between the anterior portions of the upper and lower trays.

91. A method as claimed in claim 90, wherein the step of engaging the posterior portions substantially prevents lateral movement between the upper and lower trays.

92. A method as claimed in claim 90, further comprising, prior to engaging the posterior portions, attaching the engagement features to one or both of the trays.

93. A method as claimed in claim 90, wherein the steps of installing the upper and lower trays comprises applying a substance to the interiors of the trays to enhance the attachment of the trays to the subject's teeth.

94. A method as claimed in claim 90, further comprising inserting an endotracheal tube down the subject's throat, and positioning part of the endotracheal tube between the anterior portions of the upper and lower trays.

95. A method as claimed in claim 94, further comprising fastening the endotracheal tube to the lower tray.

96. A method as claimed in claim 90, wherein the subject has at least one fractured jaw comprising fracture segments, and the step of installing the tray(s) onto the subject's dentition(s) corresponding to the fractured jaw(s) comprises initially moving the fracture segments so that adjacent bone ends of the fracture segments are generally aligned, and the installed tray(s) minimise(s) relative movement of the fracture segments.

97. A method as claimed in claim 90, wherein the upper and lower jaws are angularly misaligned, and the method comprises engaging the posterior portions without correcting the angular misalignment.
